(19) United States
(12) Patent Application Publication
(10) Pub. No.: US 2022/0354596 A1
(43) Pub. Date: Nov. 10, 2022

(54) ROBOTIC SURGERY SYSTEMS, DEVICES, AND METHODS OF USE

(71) Applicant: Levita Magnetics International Corp., Menlo Park, CA (US)

(72) Inventors: Greg LIU, Sunnyvale, CA (US); Alberto RODRIGUEZ-NAVARRO, San Francisco, CA (US); David TRASK, Redwood City, CA (US); Vicente OPASO-VALENZUELA, Santiago (CL)

(21) Appl. No.: 17/701,294

(22) Filed: Mar. 22, 2022

Related U.S. Application Data
(60) Provisional application No. 63/165,007, filed on Mar. 23, 2021.

(51) Int. Cl. A61B 34/30; A61B 46/10; A61B 90/50

(52) U.S. Cl. CPC A61B 34/30; A61B 46/10; A61B 90/50; A61B 2034/301; A61B 2034/302

(57) ABSTRACT

Described here are systems, devices, and methods useful for minimally invasive surgical procedures performed by a single operator. Methods of performing magnetic laparoscopic robotic surgery may comprise coupling an end effector to a support arm within a sterile field using an end effector connector, controlling the end effector within a body cavity of a patient, and decoupling the end effector from the support arm within the sterile field using the end effector connector. Coupling, controlling, and decoupling may all be capable of being performed by a single operator.

### CROSS-REFERENCE TO RELATED APPLICATIONS

This application claims the benefit of U.S. Provisional Application No. 63/165,007, filed Mar. 23, 2021, the content of which is hereby incorporated by reference in its entirety.

### TECHNICAL FIELD

Devices, systems, and methods herein relate to minimally invasive procedures using a robotic surgery system that may be operated by a single operator.

### BACKGROUND

Many surgical procedures are shifting toward the use of minimally invasive approaches in order to minimize the number and size of incisions that are made in a patient. Minimally invasive procedures such as endoscopic, laparoscopic, and thoracoscopic procedures may be associated with lower pain, quicker post-surgical recovery, shortened hospitalization, and reduced complications when compared to open surgical procedures. In general, minimally invasive robotic surgery is currently performed by two skilled surgeons (e.g., operators). A primary surgeon performs the surgical tasks (e.g., dissection, clipping, cutting, stapling, etc.) and a secondary surgeon assists in these functions. The primary surgeon is located at a console outside of a sterile field while the secondary surgeon is located within the sterile field in order to assist by, for example, changing the instruments (e.g., end effectors) coupled to a robotic surgery system. The secondary surgeon may assist the primary surgeon by holding an instrument in each hand such as an optical sensor (e.g., camera) and a retractor. Accordingly, it may be desirable to provide a robotic surgery system that may be less cumbersome and resource intensive than those traditionally in use.

### SUMMARY

Described here are systems, devices, and methods useful for minimally invasive surgical procedures. In some variations, the procedures described herein may be performed by a single operator absent additional assistance from another operator. Generally, an end effector connector may comprise a housing configured to receive an end effector, an arm configured to releasably couple the housing to a robot. The arm may comprise a magnetic portion, and a housing release mechanism configured to manually release the housing from the arm.

In some variations, the magnetic portion may be coupled to a proximal portion of the arm. In some variations, the magnetic portion may be configured to magnetically couple the arm to the robot through a sterile drape. In some variations, the sterile drape may be coupled between the magnetic portion and the arm. In some variations, a first side of the magnetic portion may be configured to mechanically and magnetically attach to a flange of the support arm. In some variations, the first side of the magnetic portion may comprise a channel configured to receive one or more lead wires. In some variations, the magnetic portion may comprise a robot engagement feature configured to reduce a radial shear force. In some variations, the robot engagement feature may comprise a circular projection.

In some variations, the arm may define a circular recess. In some variations, the magnetic portion may comprise a rotational alignment feature. In some variations, the rotational alignment feature may comprise a linear projection. In some variations, the arm may define a linear recess. In some variations, the magnetic portion may comprise a magnetic release mechanism configured to manually release the arm from the robot. In some variations, the magnetic release mechanism may comprise one or more levers. In some variations, one or more levers may couple to a circumference of the magnetic portion. In some variations, the magnetic release mechanism may comprise one or more cams configured to push the magnetic portion away from the arm.

In some variations, the magnetic portion may define a first longitudinal axis and the housing defines a second longitudinal axis. The first longitudinal axis and the second longitudinal axis may be non-parallel. In some variations, a first angle between the first longitudinal axis and the second longitudinal axis may be up to about 40 degrees. In some variations, the first angle may be between about 15 degrees and about 35 degrees, including all ranges and sub-values in-between. In some variations, the end effector received in the housing may define a third longitudinal axis parallel to the second longitudinal axis. In some variations, a second angle between the first longitudinal axis and the third longitudinal axis may be up to about 40 degrees, including all ranges and sub-values in-between. In some variations, the second angle may be between about 15 degrees and about 35 degrees, including all ranges and sub-values in-between. In some variations, the magnetic portion may be separated from the housing by a height of up to about 30 cm and a length of up to about 30 cm, including all ranges and sub-values in-between.

In some variations, the arm may comprise an arm handle. In some variations, the arm handle may be configured to be held by a first hand of an operator. In some variations, the magnetic portion may define a first longitudinal axis and the arm handle may define a fourth longitudinal axis. The first longitudinal axis and the fourth longitudinal axis may be non-parallel. In some variations, a third angle between the first longitudinal axis and the fourth longitudinal axis may be up to about 75 degrees. In some variations, the third angle may be between about 15 degrees and about 60 degrees. In some variations, the arm may comprise a convex shape. In some variations, a distal portion of the arm may be inferior to a proximal portion of the arm. In some variations, the arm may comprise one or more lead wires configured to electrically couple the robot to the end effector. In some variations, the arm may comprise an arm handle and the lead wires extend parallel to the arm handle.

In some variations, the housing release mechanism may be coupled to a distal portion of the arm and a proximal portion of the housing. In some variations, the housing release mechanism may comprise a first portion coupled to the arm and a second portion coupled to the housing. In some variations, the housing release mechanism may comprise a housing engagement feature configured to engage a first portion and a second portion of the housing release mechanism to each other. In some variations, the housing engagement feature may comprise a first housing engagement feature of the first portion and a second housing engagement feature of the second portion. In some variations, the first housing engagement feature may comprise a rounded projection, and the second housing engagement feature may comprise a first recess. In some variations, the housing release mechanism may comprise a rotational alignment feature configured to inhibit rotation of the first portion relative to the second portion. In some variations, the rotational alignment feature may comprise a first rotational alignment feature of the first portion and a second rotational alignment feature of the second portion. In some variations, the first rotational alignment feature may comprise a shaped projection, and the second rotational alignment feature may comprise a second recess. In some variations, the housing release mechanism may comprise a switch configured to release the housing from the arm. In some variations, one of the first portion and the second portion may comprise a switch configured to release the first portion from the second portion. In some variations, the switch may be configured to release the first housing engagement feature from the second housing engagement feature. In some variations, the switch may comprise a spring configured to engage the first housing engagement feature to mechanically couple the first portion to the second portion. In some variations, the housing engagement feature may comprise one or more of a magnet, a dovetail joint, and a living hinge. In some variations, the housing release mechanism may be superior to the end effector received in the housing. In some variations, the housing release mechanism may be inferior and distal to the magnetic portion.

In some variations, the housing may be configured to releasably couple to the end effector. In some variations, a distal portion of the housing may be configured to receive the end effector. In some variations, a proximal portion of the housing may be coupled to a lateral sidewall of the housing release mechanism. In some variations, the housing may comprise a housing handle. In some variations, the housing handle may be configured to be held by a first hand of an operator. In some variations, the housing handle may be superior relative to the end effector received in the housing. In some variations, the end effector may be received on a lateral side of the housing handle. In some variations, the housing release mechanism may be configured to be actuated by the first hand while holding the housing handle with the first hand. In some variations, the housing handle may define a handle longitudinal axis and a handle lateral axis. The housing may comprise a first stiffness along the handle longitudinal axis and a second stiffness along the handle lateral axis, the first stiffness more than the second stiffness. In some variations, the housing handle may have a width less than a length or height of the housing. In some variations, the housing may define an aperture configured for access to the end effector.

In some variations, the robot may comprise a support arm configured to moveably suspend one or more of the end effector connector and the end effector. In some variations, the support arm may comprise one or more segments coupled by one or more joints configured to provide a single degree of freedom. In some variations, the support arm may comprise one or more motors configured to translate and/or rotate the one or more joints. In some variations, the support arm may comprise six or more degrees of freedom. In some variations, the support arm may comprise less than six degrees of freedom. In some variations, the support arm may comprise one or more of an articulated robotic arm, SCARA robotic arm, and linear robotic arm. In some variations, the support arm may be mounted to a base comprising one or more of a medical cart, a patient platform, furniture, a wall, a ceiling, and a ground. In some variations, the support arm may comprise a magnetic coupler coupled to distal portion of the support arm.

In some variations, a magnetic coupler may be coupled to a distal end of the robot. The magnetic coupler may be releasably coupled to the magnetic portion of the arm with a sterile drape disposed therebetween. In some variations, the end effector may comprise one or more of a visualization device, a grasper, a retractor, a magnetic positioning device, a sensor, an intracavity device, a delivery device, a retrieval device, a stapler, a clip applier, and an electrocautery hook. In some variations, the end effector may comprise a magnetic portion.

In some variations, a sensor may be configured to detect a location of a patient body surface. In some variations, the sensor may comprise one or more of a force sensor, proximity sensor, optical sensor, motion sensor, accelerometer, gyroscope, laser rangefinder, radar, and LIDAR.

In some variations, a robotic surgery system may comprise the end effector connector. A base of the robot may be coupled to a lateral side of a patient platform.

Also described here is a method of performing magnetic laparoscopic robotic surgery comprising magnetically coupling an end effector to a support arm within a sterile field using an end effector connector, controlling the end effector within a body cavity of a patient, and decoupling the end effector from the support arm within the sterile field using the end effector connector. Coupling, controlling, and decoupling are all capable of being performed by a single operator.

In some variations, coupling and decoupling the end effector is capable of being performed by two hands of the single operator. In some variations, controlling the end effector is capable of being performed by a single hand of the single operator. In some variations, coupling and decoupling the end effector maintains the sterile field. In some variations, the sterile field may be maintained during the coupling, controlling, and decoupling steps.

Also described here is a method of decoupling an end effector from a robotic surgery system comprising providing an end effector connector coupled between the end effector, a sterile drape, and a support arm. The end effector connector may comprise an arm and a housing comprising a handle. The method may include holding the handle of the housing, and manually releasing the housing from the arm.

In some variations, holding the handle and manually releasing the housing may be simultaneously performed by a single hand. In some variations, the method may include withdrawing the handle in a direction away from a patient after releasing the housing from the arm. In some variations, the arm may comprise a magnetic portion magnetically coupled to the support arm.

Also described here is a method of coupling an end effector to a support arm comprising providing an end effector connector comprising a housing and an arm configured to be releasably coupled to the housing, magnetically coupling the arm to the support arm with a sterile drape disposed therebetween, coupling the end effector to the housing, and coupling the housing to the support arm. In some variations, coupling the housing to the arm may be performed by a single hand.

Also described here is a method of controlling a support arm comprising receiving a support arm control signal based on motion of a single foot of an operator, and controlling motion of a support arm with at least three degrees of freedom based on the received support arm control signal.

In some variations, the support arm control signal may comprise a translation motion of the support arm corresponding to a translation motion of the single foot. In some variations, the support arm control signal may comprise a lateral motion of the support arm corresponding to a lateral motion or a yaw motion of the single foot. In some variations, the support arm control signal may comprise a downward motion of the support arm corresponding to a flexion motion of the single foot. In some variations, the support arm control signal may comprise a support arm switch command corresponding to a heel movement of the single foot. In some variations, the method may comprise receiving an end effector control signal based on motion of the single foot.

In some variations, the method may comprise controlling operation of an end effector based on the end effector control signal. In some variations, the operator may be standing during the motion of the single foot.

Also described here is a support arm controller comprising a base comprising a first end and a second, the base configured to receive a midfoot of an operator. A set of forefoot switches may be coupled to the first end of the base. A set of hindfoot switches may be coupled to the second end of the base. The set of forefoot switches and the set of hindfoot switches may be configured to generate a support arm control signal.

In some variations, the support arm control signal may be configured to control a motion of the support arm, and a motion of an operator foot may correspond to the motion of the support arm. In some variations, the set of forefoot switches may comprise a first switch configured to receive a forward motion of the foot and generate the support arm control signal corresponding to forward motion of the support arm.

In some variations, the set of forefoot switches may comprise a second switch configured to receive an extension motion of the foot and generate the support arm control signal corresponding to a downward motion of the support arm. In some variations, the set of forefoot switches may comprise a third switch configured to receive a leftward motion of the foot and generate the support arm control signal corresponding to a leftward motion of the support arm. In some variations, the set of forefoot switches may comprise a fourth switch configured to receive a rightward motion of the foot and generate the support arm control signal corresponding to a rightward motion of the support arm. In some variations, the set of forefoot switches may comprise a fifth switch configured to receive a flexion motion of the foot and generate the support arm control signal corresponding to an upward motion of the support arm. In some variations, the set of forefoot switches may comprise a sixth switch configured to receive a backward motion of the foot and generate the support arm control signal corresponding to a backward motion of the support arm. In some variations, the set of forefoot switches may comprise a seventh switch configured to receive a downward motion of a hindfoot and generate a device switching signal of a robotic surgery system. In some variations, the set of forefoot switches and the set of hindfoot switches may comprise one or more of a mechanical switch, optical sensor, gyroscope, motion sensor, pressure sensor, and magnetic sensor. In some variations, the base may be elevated relative to one or more of the set of forefoot switches and one or more of the hindfoot switches.

Also described here is a method of registering an end effector comprising coupling the end effector to a support arm of a robotic surgery system in proximity to a patient. The end effector may comprise a first registration point and the patient may comprise a second registration point. The first registration point may be aligned to the second registration point. A location of the first registration point aligned to the second registration point may be registered. The location of the first registration point may be registered in a three-dimensional coordinate system of the robotic surgery system. The support arm may be controlled based on the registered first registration point.

In some variations, the second registration point may correspond to an access site of the patient. In some variations, the second registration point may correspond to an inlet of a trocar coupled to the patient. In some variations, the first registration point may intersect a longitudinal axis of the end effector. In some variations, a visual indicator of the first registration point of the end effector may be generated. In some variations, the visual indicator may comprise illumination directed at the end effector. In some variations, aligning the first registration point to the second registration point may comprise overlapping the first registration point to the second registration point.

In some variations, the registered first registration point may comprise a pivot point. In some variations, controlling the support arm may comprise one or more of pitching, yawing, and rolling the end effector within a conical range of motion comprising a vertex at the pivot point. In some variations, the conical range of motion may have a maximum cone angle θ of up to about 50 degrees. In some variations, the conical range of motion may be represented by α²+β²≤θ², where α corresponds to a pitch angle of the end effector, β corresponds to a yaw angle of the end effector, and θ corresponds to a maximum cone angle. In some variations, controlling the support arm may comprise maintaining an intersection of the end effector to the pivot point. In some variations, the second registration point may correspond to a muscle layer of an abdominal wall of the patient. In some variations, the first registration point may be reregistered to the second registration point when one or more of the patient moves relative to a patient platform, the patient platform moves, a base of a support arm is moved, and the pivot point is moved. In some variations, aligning is performed manually by an operator.

Also described here is a method of controlling an end effector may comprise measuring a force applied by a patient to an end effector coupled to a support arm, and moving the end effector relative to the patient in response to the measured force exceeding a predetermined threshold.

In some variations, the method may comprise outputting a notification in response to the measured force exceeding the predetermined threshold. In some variations, the method may comprise moving the end effector relative to the patient comprising moving the end effector away from the patient. In some variations, the method may comprise moving the end effector relative to the patient comprising moving the support arm coupled to the end effector. In some variations, the method may comprise controlling a patient platform in response to the measured force exceeding the predetermined threshold.

In some variations, the method may comprise controlling the patient platform comprising moving the patient platform away from the end effector. In some variations, the method may comprise inhibiting operator control of one or more the support arm and a patient platform in response to the measured force exceeding the predetermined threshold. In some variations, the end effector may comprise a magnetic positioning device configured to control an intracavity device disposed within the patient.

Also described here is a method of controlling an end effector comprising determining a distance between a patient and an end effector coupled to a support arm, and moving the end effector relative to the patient based on the determined distance to maintain a predetermined distance between the patient and the end effector.

In some variations, the distance may be determined using one or more of an optical sensor, a patient fiducial coupled to the patient, and an end effector fiducial coupled to the end effector. In some variations, moving the end effector relative to the patient may comprise maintaining at least a predetermined distance between the end effector and the patient.

Also described here is a method of confirming end effector decoupling comprising measuring a first force applied to a support arm using a force sensor, determining end effector removal based on the measured force exceeding a first predetermined threshold, moving the support arm away from an end effector registration point while measuring a second force applied to the support arm, and confirming end effector decoupling based on the measured second force.

In some variations, the method may comprise outputting a prompt to move the support arm away from a registration point. In some variations, the second force may be substantially no force. In some variations, the method may comprise confirming end effector coupling comprises generating an end effector decoupling signal. In some variations, moving the support arm may comprise one or more of lateral movement relative to the registration point and a rotation relative to the registration point. In some variations, moving the support arm may be inhibited if the measured second force exceeds a second predetermined threshold. In some variations, the method may comprise confirming end effector coupling based on the measured second force exceeding a second predetermined threshold.

### BRIEF DESCRIPTION OF THE DRAWINGS

FIG. 1 is a block diagram of an illustrative variation of a robotic surgery system.

FIG. 2A depicts a left side view of an illustrative variation of a robotic surgery system. FIG. 2B depicts a right side view of an illustrative variation of a robotic surgery system. FIGS. 2C, 2D, and 2E depict perspective views of an illustrative variation of a robotic surgery system.

FIG. 3A depicts a left side view of an illustrative variation of an end effector connector. FIG. 3B depicts a left side view of an illustrative variation of an end effector connector. FIGS. 3C and 3D depict perspective views of an illustrative variation of an end effector connector. FIG. 3E depicts a plan view of an illustrative variation of an end effector connector.

FIGS. 4A and 4B depict exploded perspective views of an illustrative variation of a robotic surgery system.

FIGS. 5A and 5C depict perspective views of an illustrative variation of a magnetic portion in a first configuration. FIG. 5B depicts a perspective view of an illustrative variation of a magnetic portion in a second configuration.

FIGS. 6A and 6B depict perspective views of an illustrative variation of a housing release mechanism of an end effector connector. FIG. 6C depicts a cross-sectional view of an illustrative variation of a housing release mechanism. FIG. 6D depicts a side view of an illustrative variation of a housing release mechanism.

FIG. 7A depicts a perspective view of an illustrative variation of an input device of a robotic surgery system. FIG. 7B depicts a side view of an illustrative variation of an input device of a robotic surgery system. FIG. 7C depicts a top view of an illustrative variation of an input device of a robotic surgery system. FIG. 7D depicts a perspective view of an illustrative variation of an input device of a robotic surgery system.

FIG. 8 depicts a flowchart representation of an illustrative variation of performing robotic surgery.

FIG. 9 depicts a flowchart representation of another illustrative variation of performing robotic surgery.

FIG. 10 depicts a flowchart representation of an illustrative variation of decoupling an end effector.

FIG. 11 depicts a flowchart representation of an illustrative variation of coupling an end effector.

FIG. 12 depicts a flowchart representation of an illustrative variation of performing robotic surgery.

FIG. 13 is a schematic cross-sectional view of an illustrative variation of a method of performing robotic surgery.

FIG. 14A is a schematic side view of an illustrative variation of end effector rotation. FIG. 14B is a schematic plan view of an illustrative variation of end effector rotation. FIG. 14C is a schematic front view of an illustrative variation of end effector motion. FIGS. 14D, 14E, and 14F are schematic side views of an illustrative variation of end effector motion.

FIGS. 14G and 14H are schematic perspective views of an illustrative variation of end effector movement.

FIG. 15 depicts a flowchart representation of an illustrative variation of end effector monitoring.

FIG. 16 is a schematic diagram of an illustrative variation of end effector monitoring for a robotic surgery system.

FIG. 17 depicts a flowchart representation of an illustrative variation of end effector decoupling confirmation.

FIG. 18 is a schematic diagram of an illustrative variation of an end effector decoupling confirmation process.

FIGS. 19A and 19B are schematic plan diagrams of an illustrative variation of a robotic surgery system.

### DETAILED DESCRIPTION

Described here are systems, devices, and methods for use in minimally invasive surgical procedures confirmed to be performed by a single operator absent additional assistance from another operator. For example, the systems, devices, and methods described herein may improve minimally invasive robotic surgery by: enabling single operator operation of a robotic surgery system without a second operator; reducing sterile field management using a magnetic robotic surgery system; facilitating rapid tool (e.g., end effector) exchanges; improving operator ergonomics of a robotic surgery system; enhancing single operator control of a robotic arm; facilitating rapid and accurate end effector registration; providing hands free control of a support arm; and monitoring patient safety with respect to the robotic surgery system.

While conventional robotic surgery systems require a first operator to be assisted by a less skilled second operator (e.g., scrub nurse) to perform various functions during a minimally invasive surgical procedure, the systems and methods disclosed herein may not require a second skilled operator to assist the single operator. For example, the systems, devices, and methods described herein may enable rapid end effector cleaning and port changes with just the single operator without affecting a sterile field, thereby alleviating the need for aseptic technique.

In some variations, the robotic surgery methods described herein enable single operator end effector exchange and robotic arm control for laparoscopic procedures. In some variations, a method of performing magnetic laparoscopic robotic surgery may comprise coupling an end effector to a robotic arm within a sterile field using a magnetic portion, controlling the end effector within a body cavity of a patient, and decoupling the end effector from the robotic arm within the sterile field. The coupling, controlling, and decoupling steps are performed by a single operator.

Generally, the systems and devices described herein facilitate rapid end effector exchanges with a robot arm that may improve the speed and/or efficiency of a robotic surgical procedure. In some variations, an end effector connector may comprise a housing configured to receive an end effector. The end effector may also include an arm configured to releasably couple the housing to a robot. The arm may comprise a magnetic portion, and a housing release mechanism configured to manually release the housing from the arm. For example, end effector exchange may be performed within and without change to a sterile field. Furthermore, the end effector connector may couple a sterile drape to the robot.

In some variations, the end effector connector may function as a handle held by just a single hand of the operator. For example, the end effector connector can be held and moved (e.g., hand guided) by the operator to reposition the end effector as desired. Moreover, the end effector may be released from the robot via the end effector connector to facilitate rapid tool changes and/or cleaning.

In some variations, a physical layout and/or configuration of the robotic surgery system may improve the ergonomics (e.g., geometry) between each of the robotic arm, end effector, single operator, and patient, as well as the ergonomics of the end effector disposed within the patient. In some variations, a geometry of the end effector connector may be configured to provide clearance (e.g., working space) beneath the robot for one or more of the patient, end effector, and operator. Moreover, the end effector connector may have a configuration that facilitates physical access to the end effector. Additionally or alternatively, the robotic surgery system may position the robotic arm away from the patient and improve an accessible range of an end effector coupled to the robotic arm.

In some variations, an intuitive input device (e.g., foot controller, foot pedal) may enable single operator control of one or more robotic arms and end effectors by freeing the hands and visual attention of the operator to be elsewhere. In some variations, a method of performing robotic surgery may comprise receiving a robotic arm control signal based on motion of a single foot of an operator. Foot motion may correspond to end effector motion via the robot arm. Motion of a robotic arm with at least three degrees of freedom may be controlled based on the received robotic arm control signal.

In some variations, the robotic surgery system may ensure patient safety by locating (e.g., registering) and monitoring a position of an end effector relative to an access site to ensure that input motions that would damage tissue (e.g., collision of the end effector to an incision in an abdominal wall) are inhibited. The robotic surgery system may also monitor one or more of a position of an external end effector relative to the patient, an end effector attachment status, and a position of a patient platform. In some variations, an end effector may be moved in response to a monitored change in one or more of the patient and robotic surgery system that could otherwise injure the patient. For example, an end effector may automatically move in sync with a change in height of a patient platform.

Some of the surgery systems described herein may be used to perform surgical procedures such as a cholecystectomy, appendectomy, colectomy, hernia repair, sleeve gastrectomy or other bariatric procedures, nephrectomy, hysterectomy, oophorectomy, lobectomy, salpingectomy, fallopian tubal ligation, hernia repair including inguinal and hiatal.

### I. Systems and Devices

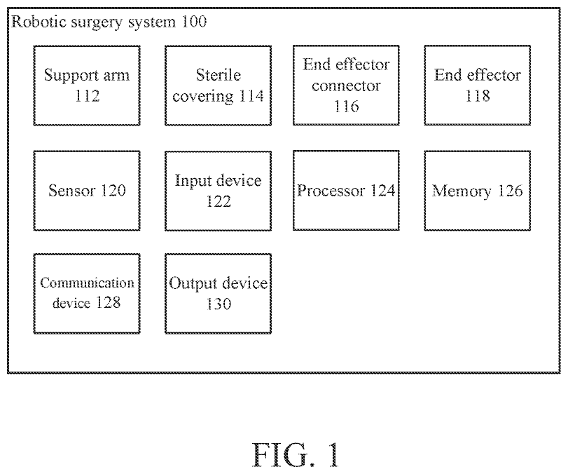

Generally, the robotic surgery systems described herein may be operated by a single operator using intuitive control schemes, improved ergonomics, and patient safety monitoring. A block diagram of an exemplary robotic surgery system 100 is depicted in FIG. 1. The system 100 may comprise one or more of a support arm 112, a sterile covering 114, an end effector connector 116, an end effector 118, a sensor 120, an input device 122, a processor 124, a memory 126, a communication device 128, and an output device 130, each of which are described in more detail herein. In some variations, the support arm 112 may be configured to moveably suspend, hold, and/or operate an end effector relative to a patient (e.g., patient on a patient platform) based on control (e.g., control inputs, manual manipulation) of a single operator. In some variations, the sterile covering 114 may be disposed between the support arm 112 and the end effector 116 to form a sterile field. For example, the sterile covering 114 may be magnetically coupled between a distal end of the support arm 112 and a proximal end of the end effector connector 116.

In some variations, the end effector connector 116 may be configured to couple the support arm 112 to the end effector 118 and facilitate single operator operation (e.g., assembly, control, disassembly). For example, the end effector 116 may facilitate single operator control through simplified and rapid end effector exchanges as well as improved surgical procedure ergonomics that may reduce procedure times and improve patient outcomes. As described in more detail herein, the end effector 118 may comprise one or more end effectors used in a surgical procedure. In some variations, the sensor 120 may comprise one or more sensors configured to measure one or more characteristics corresponding to one or more of the patient and surgery system 100 including, but not limited to, one or more of the support arm 122, the sterile covering 114, the end effector connector 116, and the end effector 118.

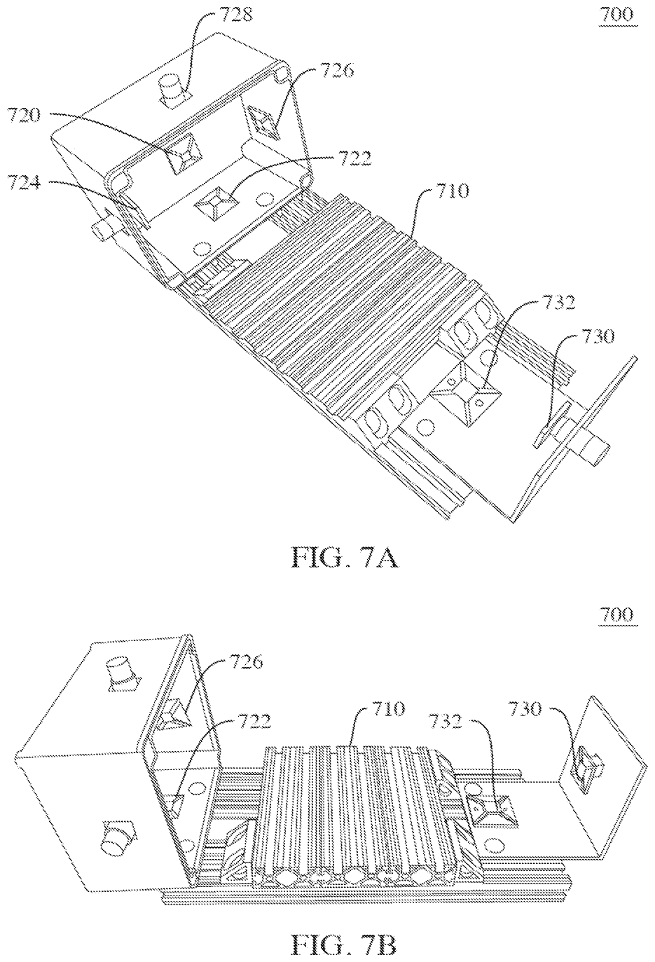

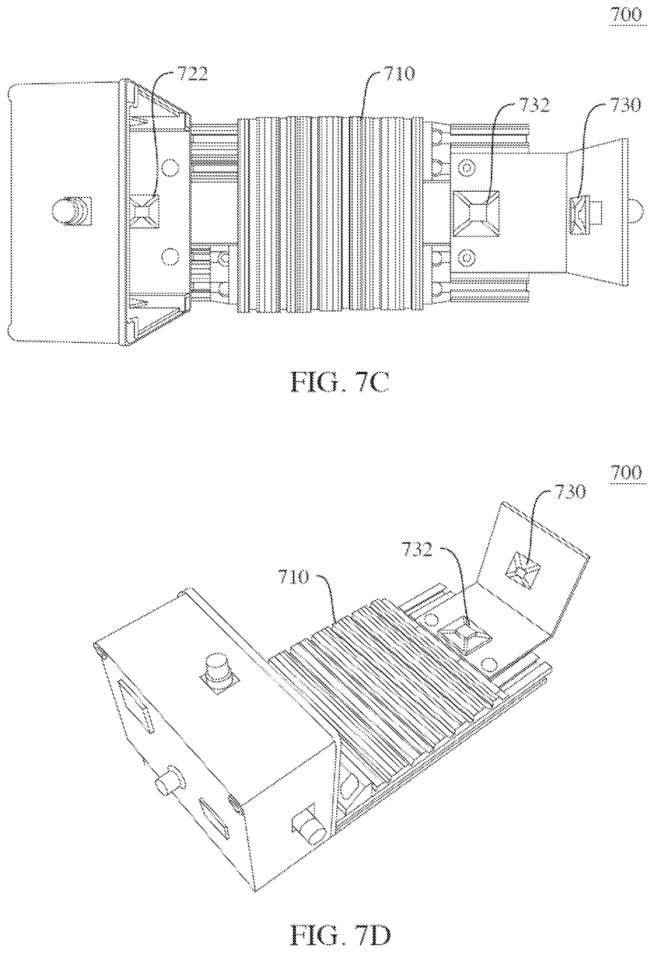

In some variations, the input device 122 may be configured to generate an input signal based on an operator input, as described in more detail with respect to FIGS. 7A-7E. In some variations, the processor 124 and memory 126 may be configured to control the surgery system 100. In some variations, the communication device 128 may be configured to communicate with one or more components of the system 100 as well as with networks and other computer systems. In some variations, the output device 130 may be configured to output data corresponding to the surgery system 100.

In some variations, a robotic surgery system may comprise a first support arm coupled to an endoscope via a first end effector connector, and a second support arm coupled to a magnetic positioning device via a second end effector connector. Each of the first support arm and the second support arm may be controlled by an operator using an input device such as input device 700 as described herein. In this manner, a single operator may independently control a set of end effectors coupled to a robotic surgery system without assistance from a second operator.

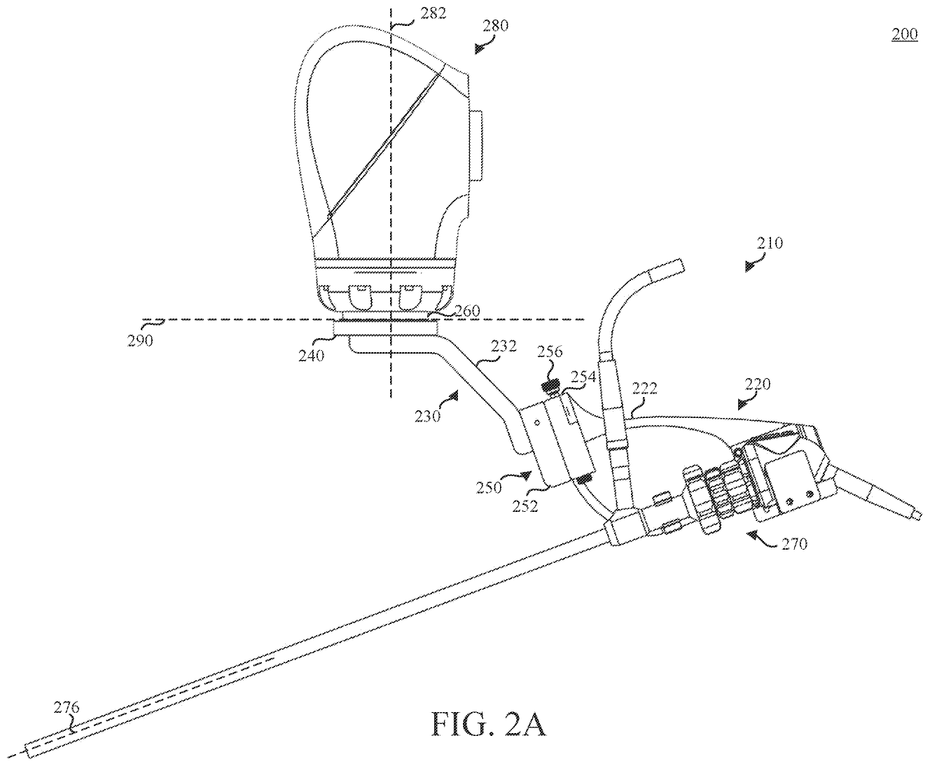

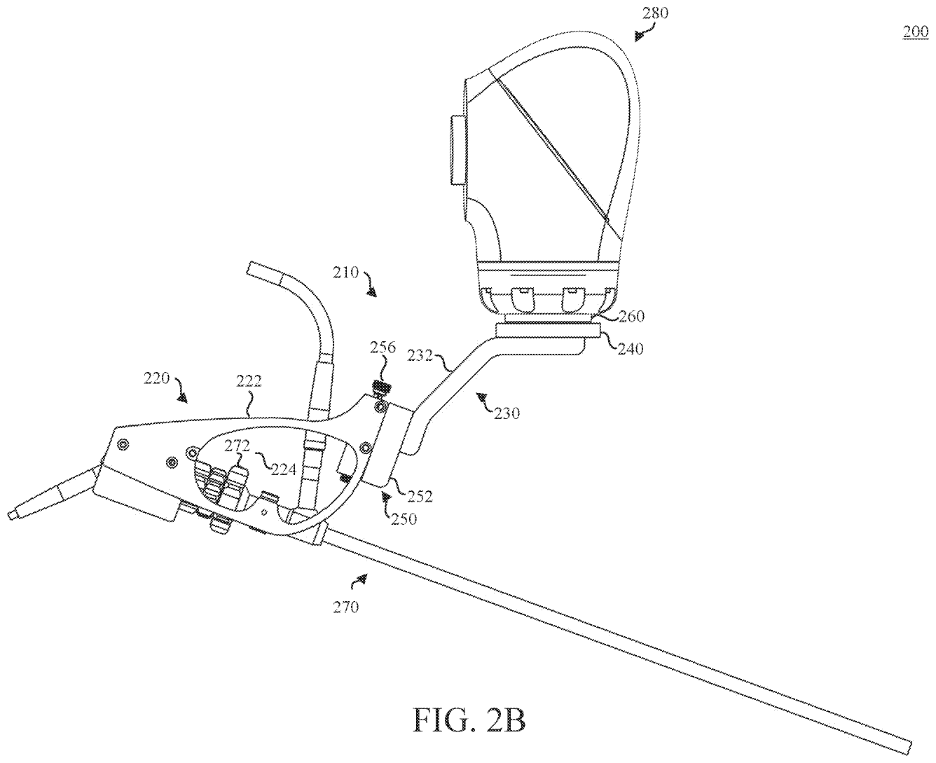

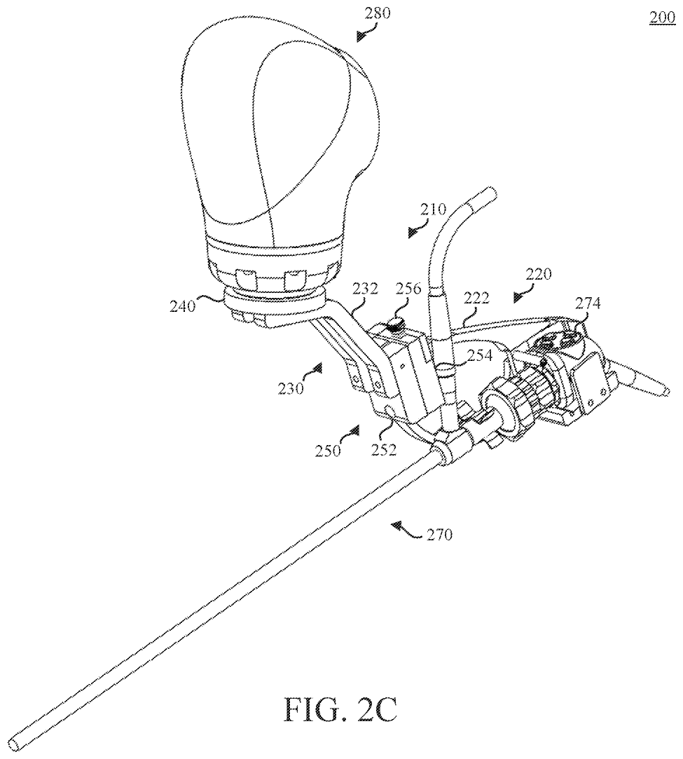

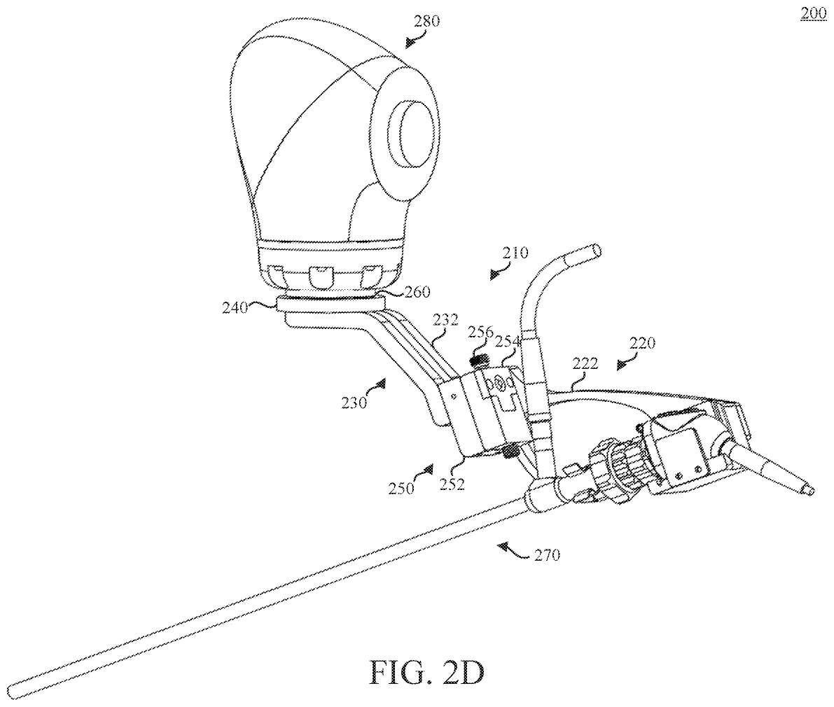

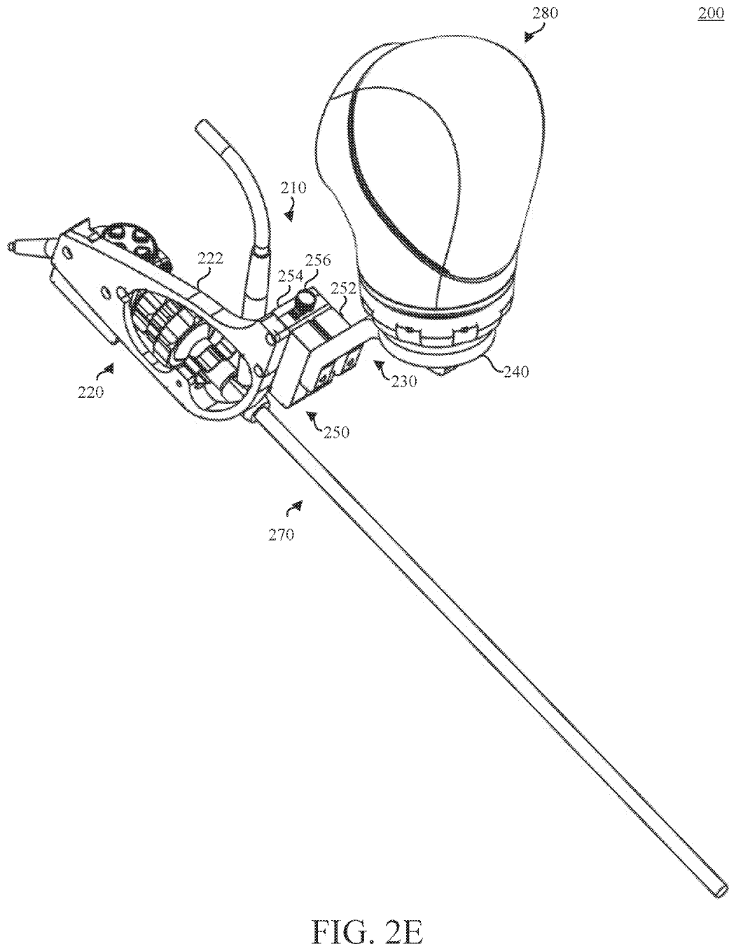

FIGS. 2A and 2B depict respective left side and right side views a robotic surgery system 200. FIGS. 2C-2E depict perspective views the robotic surgery system 200. In some variations, the system 200 may comprise one or more of an end effector connector 210, an end effector 270, and a support arm 280 (e.g., robot, robotic arm). For example, the end effector connector 210 may be configured to releasably couple the end effector 270 to a distal end of the support arm 280. In some variations, the end effector connector 210 may comprise a housing 220, an arm 230, and a housing release mechanism 250 releasably coupled therebetween.

In some variations, the housing 220 may be configured to receive the end effector 270. For example, the housing 220 may be configured to hold the end effector 270 in a predetermined position and/or orientation relative to the support arm 280. In some variations, the housing 220 may comprise a housing handle 222 configured to be held and/or manipulated by an operator (e.g., using a single hand). For example, the operator may grip the housing handle 222 with a single hand to manipulate the position and/or orientation of the end effector 270. While the support arm 280 may generally be controlled by a processor to position the end effector 270 as desired, manual manipulation by the operator holding the housing handle 232 may facilitate fine adjustments to the position and/or orientation of the end effector 270.

In FIGS. 2A-2E, the housing 220 is illustratively configured to receive an endoscope 270, although any end effector may be used. In some variations, the housing 220 may be configured to facilitate operator access to the end effector 270. As shown in FIG. 2B, the housing 210 may define an aperture 224 (e.g., cutout) configured for access to the end effector 270. The housing 220 coupled to the end effector 270 may be configured to facilitate free access to the end effector 270. For example, an operator may insert a single hand through the aperture 224 in order to manipulate a focus 272 of the endoscope 270. Additionally or alternatively, as shown in FIG. 2C, the housing 210 may be configured to facilitate access to end effector controls such as an input device 274 (e.g., camera control buttons, switch). Accordingly, the housing 220 and the end effector connector 210 may function unobtrusively to the operator. Furthermore, the housing 220 may improve the ergonomics of end effector 270 manipulation by providing a handle 222.

Generally, the shape and dimensions of the end effector connector 210 may control the positioning and/or orientation of the end effector 270 relative to the support arm 280. For example, as shown in FIG. 2A, a longitudinal axis 276 of the end effector 270 may be angled at a non-perpendicular angle relative to a longitudinal axis 282 of the support arm 280 in order to provide a favorable entry angle for an end effector 270 used in a surgical procedure. For example, an angle between a longitudinal axis 282 of the support arm 280 and a longitudinal axis 276 of the end effector may be between about 90 degrees and about 130 degrees, between about 105 degrees and about 125 degrees, including all ranges and sub-values in-between.

Moreover, a space directly below the support arm 280 (e.g., along the longitudinal axis 282) may comprise empty space absent the end effector connector 210 and the end effector 270, which may be reserved for patient anatomy (e.g., patient abdomen). This space reservation (e.g., clearance) formed by the end effector connector 220 may improve the ergonomics and safety of a surgical procedure. For example, as shown in FIG. 2A, the end effector 270 may be located below and away from the support arm 280. With respect to FIG. 2A, a patient may be disposed under and/or to the left of the support arm 280 while the end effector 270 may be disposed under and/or to the right of the support arm 280.

In some variations, a proximal portion of the arm 230 may be releasably coupled (e.g., magnetically coupled) to a distal end of a support arm 280. In some variations, the arm 230 may be configured to releasably couple to the housing 220. For example, a distal portion of the arm 230 may be coupled to a proximal portion of the housing 220. In some variations, the arm 230 may comprise a magnetic portion 240. For example, the magnetic portion 240 may be coupled to a proximal portion of the arm 230.

In some variations, the arm 230 may comprise an arm handle 232. For example, the arm handle 232 may be configured to be held and/or manipulated by an operator (e.g., using a single hand) in a manner similar to housing handle 222. Thus, the arm 230 may improve the ergonomics of end effector 270 manipulation.

In some variations, the arm 230 may comprise one or more of a convex shape and a concave shape. In some variations, the arm 230 may comprise one or more lead wires (not shown) configured to electrically couple the support arm 280 to the end effector 270. For example, an electrical connection may be formed between the support arm 280 and the end effector 270 via lead wires extending through (or in parallel with) one or more of the magnetic portion 260 and the arm 230.

In some variations, the magnetic portion 240 may be configured to magnetically couple the arm 230 to a magnetic coupler 260 of the support arm 280 with a sterile drape 290 (illustrated schematically only in FIG. 2A for the sake of clarity) disposed therebetween. That is, the sterile drape 290 may be coupled between the magnetic portion 240 and the arm 230. In some variations, the magnetic coupler 260 may be coupled to a distal end of the support arm 280. Accordingly, the sterile drape may be disposed between a distal end of the magnetic coupler 260 of the support arm 280 and a proximal end of the magnetic portion 240. The magnetic portion 240 of the arm 230 may be releasably coupled to the magnetic coupler 260 of the support arm 280, as described in more detail herein.

In some variations, a single operator may single-handedly attach and detach an end effector 270 from a support arm 280 within a sterile field and without affecting the sterile field. For example, the housing release mechanism 250 may be configured to manually attach and release the housing 220 from the arm 230. After releasing the housing 220 from the arm 230, the arm 230 may remain magnetically coupled to the support arm 280 to maintain the sterile field, and the end effector 270 may remain coupled to the housing 220 and be separately manipulated. This may facilitate rapid end effector cleanings and port changes.

Rapid end effector exchanges within the sterile filed that maintain sterility may reduce operator burden and surgical procedure times. In some variations, a housing release mechanism 250 may comprise a first portion 252, a second portion 254, and a switch 256 (e.g., trigger, release). In some variations, the housing release mechanism 250 may comprise a mechanical actuator. For example, to release an end effector 270 coupled to the support arm 280 via the end effector connector 210, an operator may grip the housing handle 222 with a first hand to hold the housing 222 and end effector 270 while simultaneously using a finger or thumb of the first hand to actuate the switch 256 to release the first portion 252 from the second portion 254. The operator may then move (e.g., pull) the housing 220 and end effector 270 in a direction away from the support arm 280 (and patient) using the first hand to complete the release of the end effector 270 from the support arm 280. Safety of the patient during an end effector change may be ensured by withdrawing the end effector 270 in a direction out of and away from the patient (e.g., parallel to a longitudinal axis of the end effector 270). Furthermore, the use of a single hand of the single operator may be more efficient than having a second operator assist. For example, a second operator provided to either press a switch or hold the handle would be redundant and would instead crowd (e.g., reduce the freedom of movement) of the first operator.

### A. Support Arm

The surgery systems described herein may comprise one or more support arms. Generally, one or more end effectors may be releasably coupled to a support arm where the support arm may be configured to moveably suspend the end effector and/or end effector connector so as to move and hold the end effector at a desired location. With the end effector suspended or held at a desired location by the support arm, an operator and/or controller may move at least a portion of the end effector externally of a patient. The support arm may be, for example, an articulated robotic arm, SCARA robotic arm, and/or linear robotic arm. The support arm may comprise one or more segments coupled together by a joint (e.g., shoulder, elbow, wrist) configured to provide a single degree of freedom. Joints are mechanisms that provide a single translational or rotational degrees of freedom. For example, the support arm may have six or more degrees of freedom. The set of Cartesian degrees of freedom may be represented by three translational (position) variables (e.g., surge, heave, sway) and by the three rotational (orientation) variables (e.g., roll, pitch, yaw). In some variations, the support arm may have less than six degrees of freedom.

The support arm may be configured to move over all areas of a patient body in up to three dimensions and may also maintain the end effector at an orientation perpendicular to a surface of the patient. The support arm may comprise one or more motors configured to translate and/or rotate the joints and move the support arm to a desired location and orientation. In some variations, the position of the support arm may be temporarily locked to fix the position of the end effector. The support arm may be mounted to any suitable object, such as a medical cart, furniture (e.g., a bed rail), a wall, a ceiling, or may be self-standing (e.g., on the ground). Additionally or alternatively, the support arm may be configured to be moved manually by, for example, a single operator without the assistance of a second person. Once manually moved by the single operator, the support arm may be locked to the manually mounted position. The support arm may be configured to carry a payload comprising the support arm, end effector, and any tissue coupled to the end effector (e.g., a gallbladder held by a grasper). In some variations, a distal end of a support arm may comprise a magnetic coupler such as magnetic coupler 260 depicted in FIGS. 2A-2E. The magnetic coupler may be coupled to an end effector connector (e.g., a magnetic portion of the end effector connector). In some variations, the magnetic coupler may be coupled to a distal end of the robot, the magnetic coupler releasably coupled to the magnetic portion of the arm with a sterile drape disposed therebetween. The magnetic coupler may be coupled to distal portion of the support arm.

In some variations, the relative positions of the patient platform, patient, operator, and support arm may be configured to aid the ergonomics of a surgical procedure. In some variations, an operator may be located on a first side of a patient platform during a surgical procedure. In variations where the support arm is mounted on a base, the base may be located on the ground and along a second side of a patient platform adjacent the first side of the patient platform in order to maximize a range of the support arm. The first side of the patient platform may be perpendicular to the second side of the patient platform. For example, the support arm may extend from its base above and over (e.g., across) a patient disposed on the patient platform. The base may be located closer to a mid-point of the second side of the patient platform rather than an intersection of the first side and the second side in order to maximize the flexibility of the support arm to reach an access site of a patient. In some variations, a base of the robot (e.g., support arm) may be coupled to a lateral side of a patient platform.

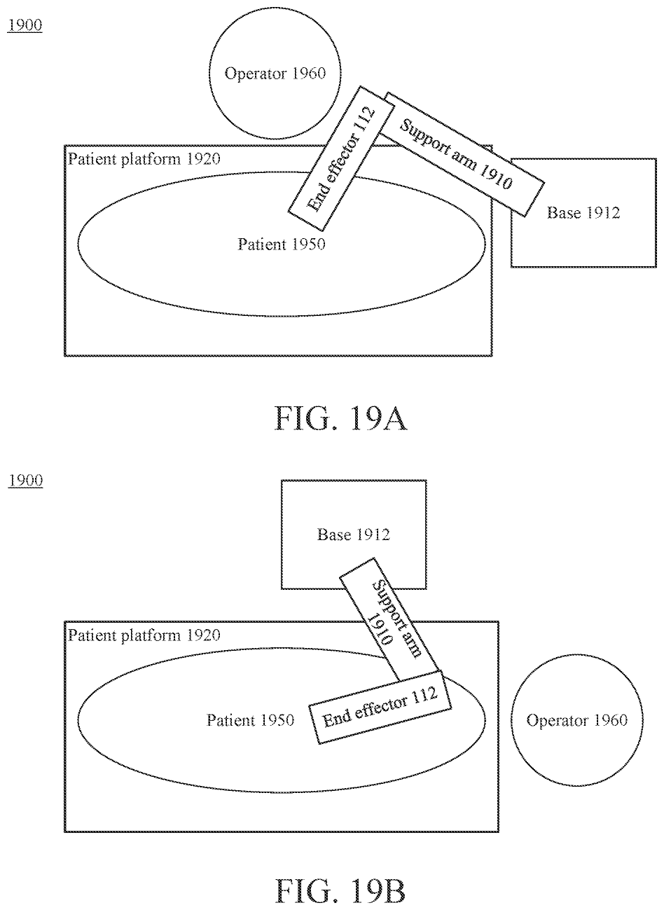

FIGS. 19A and 19B are respective schematic plan views of a robotic surgery system 1900 comprising a support arm 1910 and a corresponding base 1912 disposed on a first side of a patient platform 1920. A patient 1950 may be disposed on the patient platform 1920 and an operator 1960 may be located on a second side of the patient platform 1920 where the first side may be adjacent to the second side. From the first side of the patient platform 1920, the support arm 1910 may be configured to extend above and across the patient 1950. Accordingly, the robotic surgery system may position the robotic arm and base away from the operator and improve an accessible range of an end effector.

### B. Sterile Covering

The surgery systems described herein may comprise one or more sterile coverings (e.g., sterile drape) configured to create a sterile barrier around portions of the surgery system. In some variations, the surgery system may comprise one or more sterile coverings to form a sterile field. For example, a sterile covering may be placed between the support arm and the patient, forming a barrier between a sterile side including the patient, end effector, end effector connector, and operator and a non-sterile side including the support arm. Additionally or alternatively, one or more components of the system may be sterilizable. The sterile covering may, for example, comprise a sterile drape configured to cover at least a portion of a system component.

For example, a sterile covering (e.g., sterile bag) may be configured to create a sterile barrier with respect to a support arm. In some variations, the sterile bag may be clear and allow an operator to visualize and manually manipulate a position of the end effector by, for example, an operator grabbing a handle of a support arm or a handle attached to the end effector through the sterile bag. The sterile covering may conform tightly around one or more system components or may drape loosely so as to allow components to be adjusted within the sterile field (e.g., attachment and release of an end effector from a support arm via an end effector connector).

### C. End Effector Connector

Generally, the end effector connectors described herein may be configured to releasably connect an end effector to a support arm. The end effector connectors described herein may be configured to facilitate rapid single operator operation and/or exchange of an end effector coupled to a support arm, thereby enabling single operator operation without a second operator so as to improve operator ergonomics, and reduce sterile field management and procedure times.

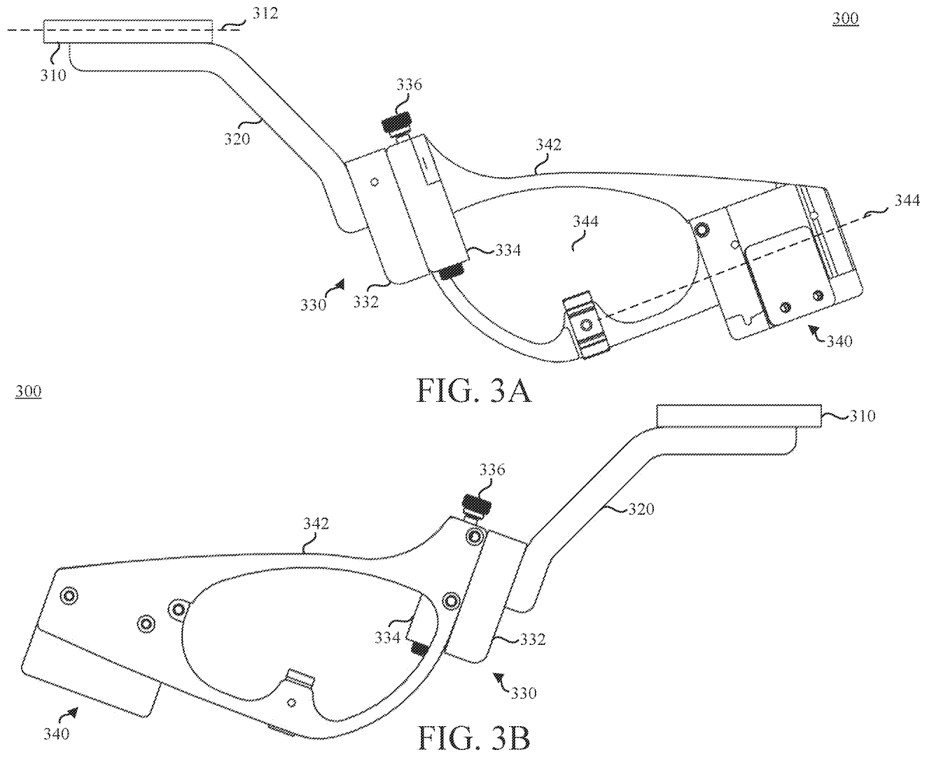

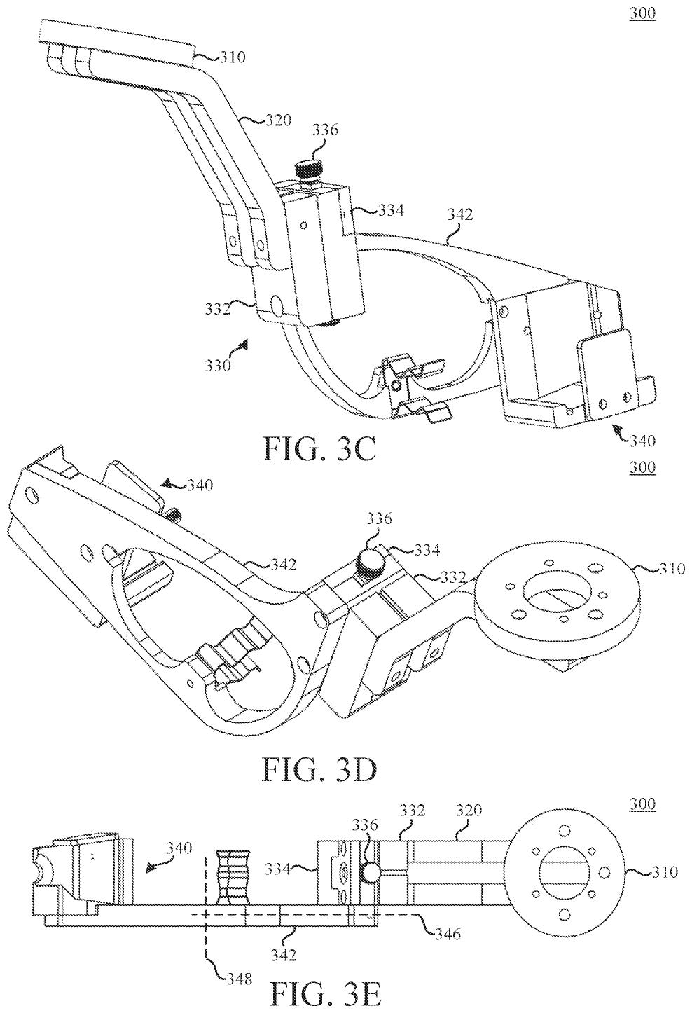

FIGS. 3A and 3B depict respective left side and right side views of an end effector connector 300. FIGS. 3C and 3D depict perspective views and FIG. 3E depicts a plan view of the end effector connector 300. In some variations, the end effector connector 300 may comprise a magnetic portion 310, an arm 320, a housing release mechanism 330, and a housing 340 comprising a housing handle 342. In some variations, a magnetic portion 310 may be coupled to a proximal portion of an arm 320. The arm 320 may have a predetermined angle with respect to the magnetic portion 310 and comprise a distal portion coupled to the housing release mechanism 330. The housing release mechanism 330 may comprise a first portion 332, a second portion 334, and a switch 336 (e.g., trigger, release) and may operate in a similar manner as described with respect to end effector connector 210. The first portion 332 may be coupled to a distal portion of the arm 320 and the second portion 334 may be coupled to a proximal portion of the housing handle 342. In some variations, the switch 336 may be configured to release the first portion 332 from the second portion 334 such that the arm 320 is releasably coupled to the housing handle 342. For example, an operator holding the housing handle 342 with just a first hand may simultaneously actuate a housing release mechanism (e.g., switch 336) with the first hand to release the housing handle 342 (and any end effector coupled thereto) from the arm 320 (and any support arm coupled thereto). In some variations, the housing handle 342 may be superior relative to an end effector received in the housing 340. The end effector may be received on a lateral side of the housing handle 342. In some variations, the switch 336 may be disposed on either of the first portion 332 and the second portion 334. In some variations, the switch 336 may be disposed on a side wall of the housing release mechanism 330 rather than on a top wall.

An aperture 344 defined by the housing 340 may be configured to allow an operator to form a substantially closed first around the housing handle 342 for a secure grip of the housing handle 342 and any end effector coupled thereto. In some variations, the housing handle 342 may extend away from the magnetic portion in generally the same direction as the arm 320. In some variations, housing 340 may be configured to receive and hold an end effector (not shown for the sake of clarity). In some variations, a proximal portion of the housing may be coupled to a lateral sidewall of the housing release mechanism. For example, as shown in FIG. 3D, a proximal portion of the housing handle 342 may be attached to a right side of the second portion 334 of the housing release mechanism 330 to aid ergonomics for a right-handed operator. Alternatively, the proximal portion of the housing handle 342 may be attached to a left side of the second portion 334 of the housing release mechanism 330 to aid ergonomics for a left-handed operator.

### D. Magnetic Portion

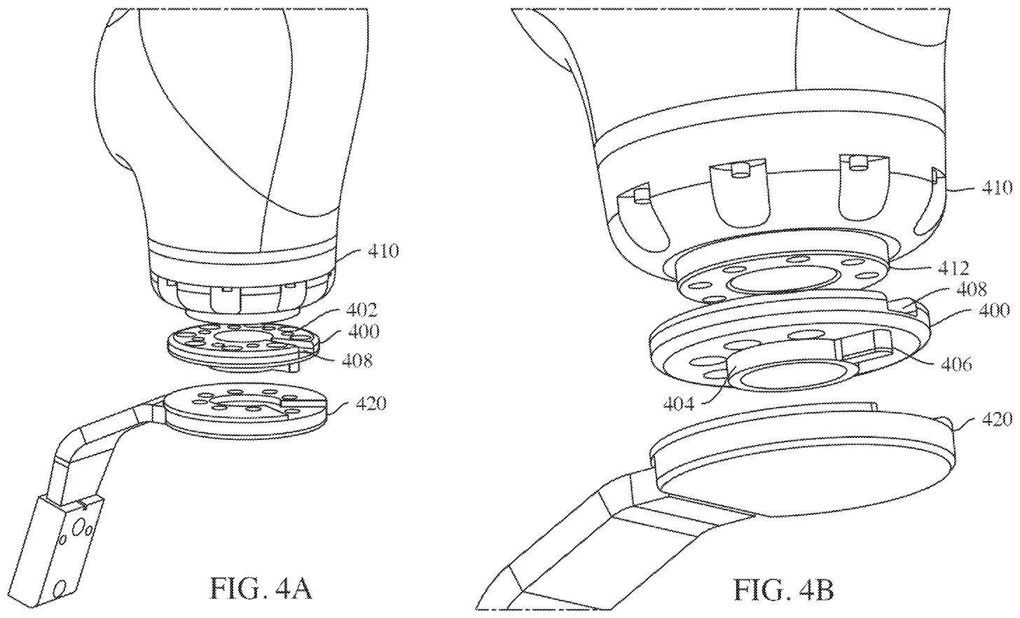

Generally, a magnetic portion of an end effector connector described herein may be configured to magnetically couple one or more of an end effector and a sterile drape to a support arm. The magnetic portion may facilitate an easy, predictable, and secure attachment and release process between an end effector and a support arm. For example, FIGS. 4A and 4B depict exploded perspective views of a magnetic portion 400 between a support arm 410 and an end effector connector 420. In some variations, the magnetic portion 400 may comprise one or more magnets 402. In some variations, the magnetic portion 400 may comprise one or more of a robot engagement feature 404 and a rotational alignment feature 406.

In some variations, a first side of the magnetic portion 400 (i.e., side facing the support arm 410) may be configured to couple (e.g., attach, mechanically interlock) to the support arm 410. For example, the a first side of the magnetic portion 400 may be configured to mechanically and/or magnetically attach to a flange 412 (e.g., magnetic coupler) of the support arm 410. In some variations, a second side of the magnetic portion 400 (i.e., facing the end effector connector 420) may be configured to magnetically couple to a proximal portion of the support arm 410. In some variations, a sterile drape (not shown for the sake of clarity) may be disposed and secured between the magnetic portion 400 and the end effector connector 420 via the magnetic coupling between the magnetic portion 400 and the end effector connector 420.

The magnets 402 of the magnetic portion 400 may be configured to generate an axial attractive force to magnetically couple (e.g., clamp, attach) the magnetic portion 400 to each of the flange 412 and end effector connector 420.

In some variations, the robot engagement feature 404 and the rotational alignment feature 406 may ensure magnetic coupling between the magnetic portion 400 and the end effector connector 420. In some variations, the robot engagement feature 404 may be configured to reduce radial shear forces applied to the magnetic portion 400 (e.g., applied by the end effector connector 420). For example, the robot engagement feature 404 may comprise a circular projection disposed on the second side of the magnetic portion 400. The circular projection may be configured to mate with a circular recess of the arm of the end effector connector 420. In some variations, the rotational alignment feature 406 may be configured to reduce a rotational force applied to the magnetic portion 400 (e.g., via the end effector connector 420). For example, the rotational alignment feature 406 may comprise a linear projection extending disposed on the second side of the magnetic potion 400. The linear projection may be configured to mate with a linear recess of the arm of the end effector connector 420. In some variations, a first side of the magnetic portion 400 may comprise a channel 408 configured to receive one or more lead wires (not shown) configured to electrically couple the support arm 410 to the end effector (not shown). For example, an electrical connection may be formed between the support arm 410 and the end effector via lead wires disposed in channel 408.

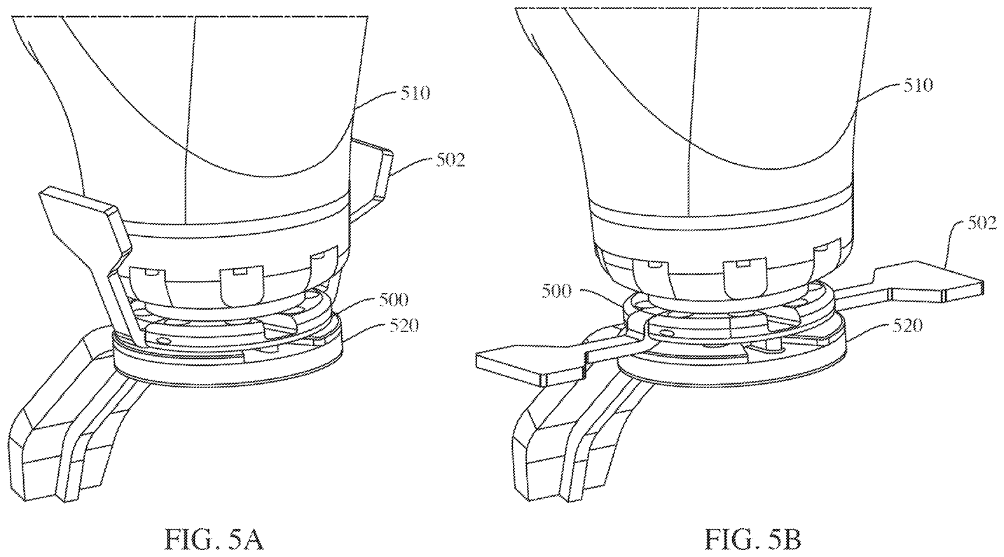

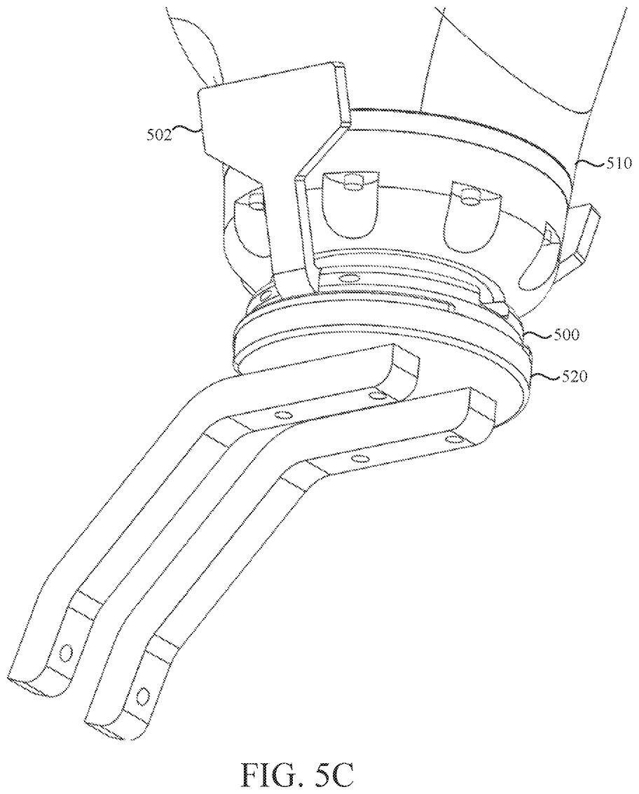

FIGS. 5A, 5B, and 5C depict perspective views of a magnetic portion 500 in a respective first configuration and second configuration. The magnetic portion 500 may be coupled between a support arm 510 and an end effector 520. The magnetic portion 500 may comprise similar elements to the magnetic portion 400 and is not repeated for the sake of brevity. In some variations, the magnetic portion 500 may comprise a magnetic release mechanism 502 configured to manually release the arm 520 from the support arm 510. For example, the magnetic release mechanism 502 may be configured to be manually actuated (e.g., by an operator) to physically separate the magnetic portion 500 from the end effector connector 520 such that the release mechanism is not itself necessarily magnetic. In some variations, the magnetic release mechanism 502 may comprise one or more levers.

In the first configuration of the magnetic release mechanism 502 shown in FIGS. 5A and 5C, the end effector connector 520 may be magnetically coupled to the magnetic portion 500. In the second configuration of the magnetic release mechanism 502 shown in FIG. 5B, the magnetic release mechanism 502 pushes the end effector connector 520 away from the magnetic portion 500 to a degree sufficient to release the magnetic coupling between the magnetic portion 500 and the end effector connector 520. For example, the levers 502 may comprise one or more cams configured to push the magnetic portion 500 of the end effector connector 520 away from the support arm 510 to facilitate detachment of the end effector connector 520 from the arm of the end effector connector 520. One or more of the levers may couple to a circumference of the magnetic portion 500.

### E. Arm

Generally, an arm of an end effector connector described herein may be configured to connect a magnetic portion to a housing release mechanism. As shown in FIGS. 2A-3E, an arm may extend at an angle away from the magnetic portion (and support arm) to provide clearance between the support arm, a patient (e.g., patient abdomen), and operator. In some variations, the angle and length of the arm may add clearance between a support arm and an operator (e.g., create more working space), and increase a range of motion of an end effector. In some variations, the arm may comprise a handle configured to be held by a single operator (e.g., by a single hand).

### F. Housing Release Mechanism

Generally, the housing release mechanisms described herein enable an arm of an end effector connector to be releasably coupled to a housing of the end effector connector. The housing release mechanism may be configured to facilitate single operator attachment and release of an end effector to a support arm via a separable (e.g., releasable) end effector connector. Once an end effector is released from a support arm, another end effector held within a corresponding housing may be coupled to the support arm via the arm using the housing release mechanism. Accordingly, end effectors may be manually exchanged from a support arm by a single operator quickly and intuitively.

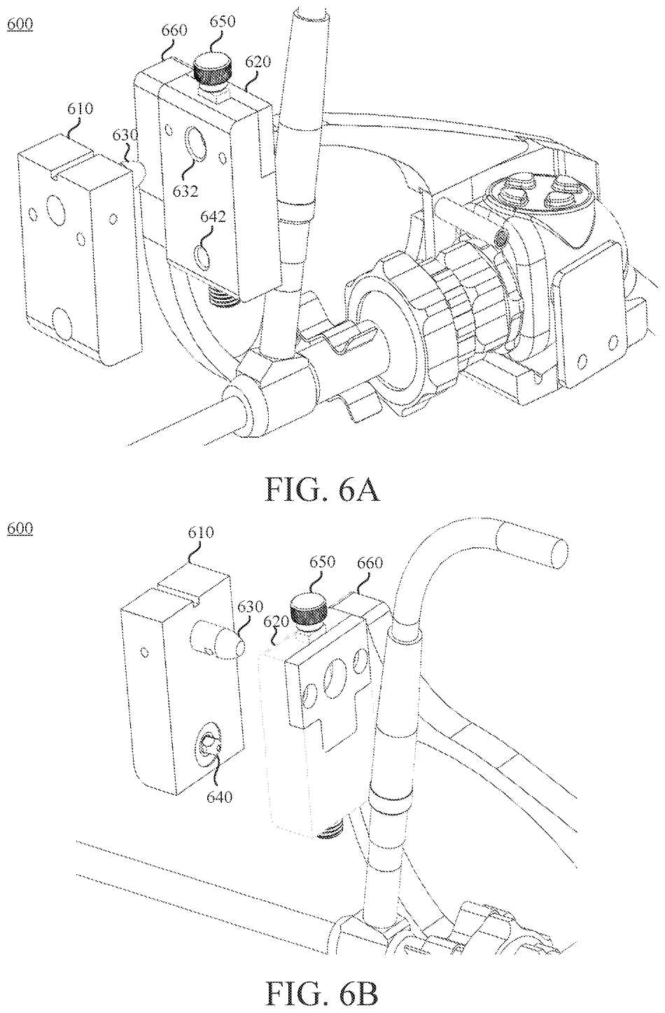

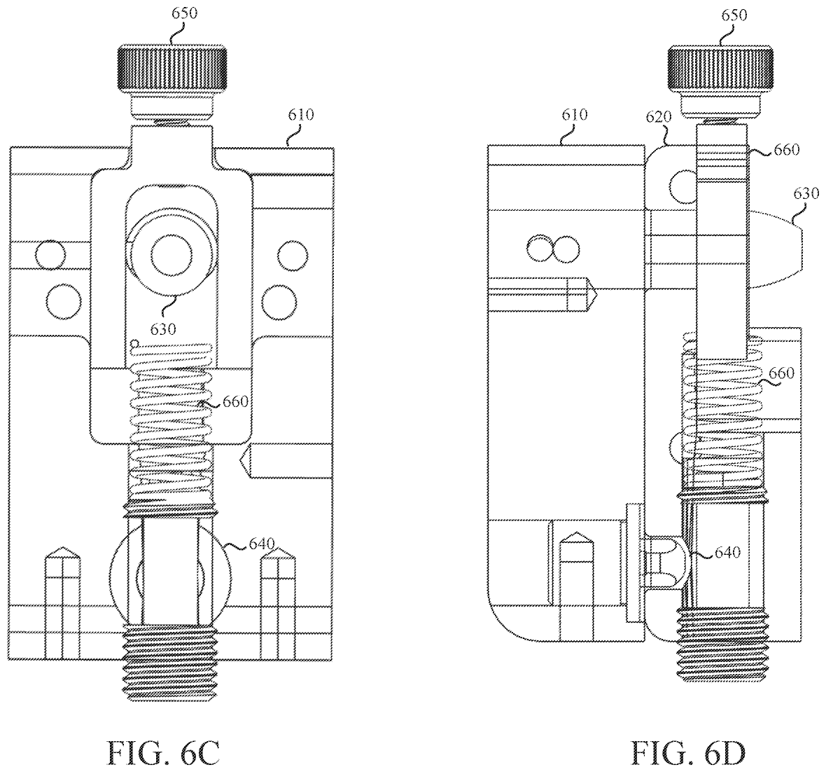

FIGS. 6A and 6B depict perspective views of a housing release mechanism 600 of an end effector connector. In some variations, the housing release mechanism 600 may be coupled to a distal portion of an arm (not shown in FIGS. 6A and 6B for the sake of clarity) and a proximal portion of a housing 660. In some variations, the housing release mechanism 600 may comprise a first portion 610 and a second portion 620. In some variations, the first portion 610 may be coupled to the arm and the second portion 620 may be coupled to the housing 660.

In some variations, the housing release mechanism 600 may comprise a housing engagement feature configured to engage a first portion 610 and a second portion 620 of the housing release mechanism 600 to each other. In some variations, the housing release mechanism 600 may comprise a rotational alignment feature configured to inhibit rotation of the first portion 610 relative to the second portion 620. For example, the first portion 610 may comprise a first housing engagement feature 630 and a first rotational alignment feature 640. The second portion 620 may comprise a second housing engagement feature 632 and a second rotational alignment feature 642. The second housing engagement feature 632 may be configured to receive and mate with the first housing engagement feature 630, and the second rotational alignment feature 642 may be configured to receive and mate with the second housing engagement feature 640. The housing engagement feature may engage the first portion 610 to the second portion 620 while the rotational engagement feature may inhibit rotation of the first portion 610 relative to the second portion 620.

In some variations, the first housing engagement feature 630 may be self-guiding (e.g., self-centering) in that the operator has a margin for error to find and advance the first housing engagement feature 630 into the corresponding second housing engagement feature 632. For example, in FIG. 6B, the first housing engagement feature 630 may comprise a rounded projection (e.g., dowel, pin) and the first rotational alignment feature 640 may comprise a shaped projection (e.g., dowel, pin). In FIG. 6A, the second housing engagement feature 632 may comprise a first recess and the second rotational alignment feature 642 may comprise a second recess. Although not shown in FIGS. 6A and 6B, the first portion 610 may be coupled (e.g., attached) to a distal portion of an arm of an end effector connector.

In some variations, one of the first portion 610 and the second portion 620 may comprise a switch 650 (e.g., trigger, release) configured to release the housing 660 from the arm. In some variations, a finger or thumb may be used to actuate the switch 650 of the first housing release mechanism 630 to release the first portion 610 from the second portion 620. For example, engaging the switch 650 may release the first housing engagement feature 630 from the second housing engagement feature 632, and thus release a housing of an end effector connector from an arm of an end effector connector.

FIG. 6C depict a cross-sectional view and FIG. 6D depicts a side view of a housing release mechanism 600. In some variations, the second portion 620 may comprise a spring 660 configured to engage the first housing engagement feature 630 to mechanically couple the first portion 610 to the second portion 620. When the switch 650 is depressed, the spring 660 may be compressed such that the first housing engagement feature 630 may easily withdraw from the second portion 620.

In some variations, the housing engagement feature of a housing release mechanism may comprise one or more of a magnet, a dovetail joint, a latch, and a living hinge. For example, a living hinge may comprise one or more of a buckle, clasp, and fastener. In some variations, at least a portion of the housing release mechanism 600 may be superior to the end effector received in the housing 660. In some variations, the housing release mechanism 600 may be inferior and distal to a magnetic portion.

### G. Housing

Generally, a housing of an end effector connector described herein may be configured to receive and hold an end effector for positioning and/or manual manipulation. For example, the housing may comprise a handle configured to be held by a single operator (e.g., by a single hand). The housing may be configured to releasably couple to the end effector. In some variations, the housing may facilitate manual manipulation of the end effector by the single operator. For example, the housing 340 may define an aperture 344 configured for access to an end effector (not shown for the sake of clarity).

In some variations, a distal portion of the housing may be configured to receive an end effector. For example, the end effector may be placed at a distal end of the housing 340 longitudinally offset from a proximal end of the end effector connector which may add clearance between a support arm and an operator (e.g., create more working space), and increase a range of motion of an end effector relative to an access site (e.g., trocar) of a patient. By contrast, an end effector attached directly to the support arm will have an increased risk of the support arm colliding with (and damaging) the patient as the end effector is moved into and out of the body.

As shown in FIG. 3A, the magnetic portion 310 defines a first longitudinal axis 312 and the housing 340 defines a second longitudinal axis 344 such that the first longitudinal axis 312 and the second longitudinal axis 344 are non-parallel. As one example, the first longitudinal axis 312 may be parallel to ground (e.g., substantially parallel to a patient platform), and the housing 340 may be angled relative to the patient platform at an advantageous angle for an end effector to access an access site (e.g., port) and a body cavity of a patient.

In some variations, an angle between the first longitudinal axis 312 and the second longitudinal axis 344 may be up to about 40 degrees, including all ranges and sub-values in-between. For example, the angle between the first longitudinal axis 312 and the second longitudinal axis 344 may be between about 15 degrees and about 35 degrees. In some variations, the magnetic portion 240 may be separated from the housing 220 by a height of up to about 30 cm and a length of up to about 30 cm. The separation height and distance between the magnetic portion 240 and housing 220 may add clearance between a support arm and an operator (e.g., create more working space), and increase a range of motion of an end effector relative to an access site (e.g., trocar) of a patient.

As shown in FIG. 3E, the housing handle 342 may define a handle longitudinal axis 346 and a handle lateral axis 348. The housing 340 may comprise a first stiffness along the handle longitudinal axis 346 and a second stiffness along the handle lateral axis 348. The first stiffness may be more than the second stiffness such that the housing 340 may be vertically stiff and laterally compliant. In some variations, lateral compliance (e.g., motion damping, flex) of the housing 340 may dampen lateral forces inadvertently applied by an end effector to an access site of a patient, thereby reducing patient injury. In some variations, the housing 340 may comprise one or more compliant portions configured to dampen a predetermined force (e.g., vibrations, lateral force). For example, lateral compliance may reduce shaking and may enable improved visualization when the end effector is an endoscope. In some variations, one or more of a thickness of the housing handle 342 and size of an aperture 344 may correspond to a lateral compliance of the housing 340. In some variations, the housing handle 342 may have a width less than a length or height of the housing 340.

### H. End Effector

Generally, the end effectors described herein are not particularly limited and may comprise one or more of a visualization device, a grasper, a retractor, a magnetic positioning device, a sensor, an intracavity device, a delivery device, a stapler, a clip applier, an electrocautery hook, and other surgical instrument that may be advanced in a minimally invasive manner through an access site. In some variations, the end effector may comprise a magnetic portion.

In some variations, an end effector (e.g., visualization device, intracavity device) may be configured to be introduced into a body cavity or lumen through an access site such as a trocar or other suitable port, or through a natural orifice. The end effectors advanced into the body cavity or lumen through an access site may be advanced such that the end effector does not block the introduction and/or retrieval of other end effectors using the access site. Thus, a plurality of end effectors may be disposed and actuated within a patient body cavity or lumen.

The end effectors may be configured to be attracted to one or more magnets positioned externally of the body to move, reposition, and/or hold the intracavity device (which may in turn provide traction for tissue held by or otherwise in contact with the intracavity device). Accordingly, at least a portion of the intracavity devices described herein may be formed from or otherwise include one or more metallic or magnetic materials which may be attracted to a magnetic field. The materials may include one or more magnetic or ferromagnetic materials, such as, for example, stainless steel, iron, cobalt, nickel, neodymium iron boron, samarium cobalt, alnico, ceramic ferrite, alloys thereof and/or combinations thereof. The magnetic portion of the intracavity device may thus be attracted to a magnetic field produced by an external magnetic positioning device. Furthermore, in some variations, the magnetic portion of the intracavity device may allow coupling to a delivery device, as described in more detail herein.

The end effectors may be used within any suitable body cavity or lumen such as but not limited to the abdominal cavity, thoracic cavity, stomach, or intestines. The end effectors advanced into a body cavity or lumen may perform a number of functions and are described in detail herein.

In some variations, an end effector may comprise a visualization device (e.g., endoscope) configured to be visualize a desired field of view during a minimally invasive procedure. In some variations, an end effector may comprise a grasper used to grasp, retract or otherwise provide remote manipulation and/or traction to tissue. In particular, magnetically controlled graspers may be advanced into a patient and releasably engage tissue. Graspers suitable for use in the surgery systems here are described in U.S. patent application Ser. No. 14/019,370, filed Sep. 5, 2013, and titled “Grasper with Magnetically-Controlled Positioning,” U.S. patent application Ser. No. 15/195,898, filed Jun. 28, 2016, and titled “Laparoscopic Graspers and Systems Therefor,” U.S. patent application Ser. No. 13/132,185, filed Aug. 17, 2011, and titled “Remote Traction and Guidance Systems for Mini-Invasive Surgery,” and International Patent Application No. PCT/US2016/027390, filed Apr. 13, 2016, and titled “Grasper with Magnetically-Controlled Positioning,” each of which is hereby incorporated by reference in its entirety.

In some variations, an end effector may comprise a retractor described used to retract or otherwise support and/or move internal organs of a patient. In particular, magnetically controlled retractors may be advanced into a patient and retract tissue to displace it from a surgical site inside the patient and/or otherwise engage tissue to increase surgical access to that tissue. Furthermore, the retractors may be configured to be maintained in position without requiring a handle or grasper. For example, in some variations, a retractor may be configured to form a sling to retract tissue. The terminal ends may comprise a magnetic material or have magnetic masses disposed on them, such that they are configured to be attracted to a magnetic field. When a portion of the retractor is looped underneath a portion of tissue, at least a portion of the tissue may be suspended by the retractor and moved towards the patient wall. In some variations, the retractor may be configured to transition between a substantially linear configuration and the curvilinear configuration.

Other retractors suitable for use in the surgery systems here are described in International Patent Application No. PCT/US2016/027385, filed Apr. 13, 2016, and titled “Retractor Systems, Devices, and Methods for Use,” which is hereby incorporated by reference in its entirety. Other suitable retractors may include, for example, one or more of a coiled retractor, cradle retractor, lever retractor, platform retractor, and J-hook.

### I. Delivery Device

The delivery devices described herein are generally configured to releasably carry one or more intracavity devices. A delivery device may be used to deliver one or more intracavity devices into a body cavity or lumen. Because the delivery devices may be releasably coupled to the intracavity devices, the delivery devices may be removed from the body cavity after delivery of the intracavity device, which may keep the access site (e.g. trocar or natural orifice) free for the delivery of other intracavity devices or other tools. In some instances, the delivery device may be configured to re-couple to the intracavity device to reposition or remove the intracavity device from a body cavity or lumen. In other instances, the system may comprise a separate retrieval device configured to reposition or remove the intracavity device from a body cavity or lumen. In some variations, the delivery device or retrieval device may be further configured to actuate an intracavity device.

When the intracavity device is a grasper, the delivery devices described here may be configured to releasably carry a grasper, and may be further configured to actuate the grasper to selectively connect the grasper to tissue or release the grasper from tissue. The delivery devices may be typically further configured to release the grasper from the delivery device (e.g., after the grasper has been connected to tissue). In some instances, the delivery device may be configured to re-couple to the grasper to reposition or remove the grasper from a body cavity or lumen. In other instances the system may comprise a separate retrieval device configured to reposition or remove the grasper from a body cavity or lumen. In some instances, the delivery device or retrieval device may be used with the grasper to remove tissue from the body. For example, the grasper may be connected to a tissue such as a gall bladder, the tissue may be severed from the body (e.g., using one or more surgical tools), and the grasper may be retrieved using the delivery device or another retrieval device to remove the grasper and tissue from the body.

Delivery devices suitable for use in the surgery systems here are described in U.S. patent application Ser. No. 14/019,370, filed Sep. 5, 2013, and titled “Grasper with Magnetically-Controlled Positioning,” which was previously incorporated by reference in its entirety.

It should be appreciated that while delivery devices are described herein primarily with reference to use with a grasper, the delivery devices described herein may also be used to reversibly couple to another intracavity device to deliver, position and reposition, and/or remove another intracavity device. For example, in some instances the delivery devices may be used to deliver, position and reposition, and/or remove a visualization device, such as a camera and/or light source.

### J. Magnetic Positioning Device

The surgery systems described herein may comprise one or more external magnetic positioning devices comprising an external magnet, support arm, and/or sensors. The external magnets may generate a magnetic field configured to attract one or more intracavity devices. By controlling the position and/or strength of the external magnets and thereby the position and/or strength of the magnetic fields, the external magnets may control the position of the intracavity devices disposed within a body cavity or lumen of a patient. This may free space at an access site (e.g., port) of the patient to allow additional intracavity devices to be advanced into the patient and reduce, if not eliminate, the need for a second operator such as a skilled surgeon.

The external magnets may be configured to generate a magnetic field, such that when the external magnet is positioned near a patient, a magnetic field may be generated inside the patient. This magnetic field may apply a force to and manipulate an intracavity device. In some variations, the external magnet may comprise one or more permanent magnets, one or more electromagnets, and/or one or more electropermanent magnets. Permanent magnets may be formed from suitable magnetic and ferromagnetic materials such as, but not limited to, rare-earth magnets (e.g., samarium-cobalt magnets, neodymium magnets), cobalt, gadolinium, iron, nickel, alnico alloys, ferrites, alloys thereof, combinations thereof, and the like. The external magnets may comprise any number of individual magnets, which in some instances may be formed in an array. The external magnets may have any suitable size and shape, such as cylindrical shape having a circular, oval, or semi-circle cross-section, a bar magnet having a rectangular or triangular cross section, a spherical magnet, or the like. In some variations, the external magnets may comprise permanent magnets, while in other variations, the external magnets may comprise electromagnets or electropermanent magnets. When the external magnets comprise electromagnets or electropermanent magnets, the current may be manipulated to change the strength of the external magnets and/or to turn them on/off. For example, an increase in the magnetic field generated by the external magnet may bring an intracavity device in contact with a body cavity wall of a patient while a decrease in the magnetic field may reposition the intracavity device away from the body cavity wall. Additionally, a stronger magnetic field may be needed to magnetically couple the intracavity device with the external magnet through a thick body cavity wall (e.g., a thick abdominal wall), whereas a weaker magnetic field may be desirable to reduce the attractive force between the intracavity device and the external magnet through a thin body cavity wall (e.g., a thin abdominal wall).

When an external magnetic positioning device is magnetically coupled to an intracavity device, movement of the external magnet via movement of the support arm may in turn move the intracavity device disposed within a body cavity or lumen of the patient. For example, coronal movement of the external magnet relative to the patient may result in a corresponding coronal movement of the intracavity device. As another example, moving the external magnet closer to the intracavity device using the support arm may increase the attraction between the external magnet and the intracavity device so as to bring the intracavity device in contact with a patient cavity wall, while moving the external magnet further away from the intracavity device may reduce the magnetic attraction and reposition the intracavity device away from the body cavity wall. Thus, by controlling the strength of the external magnet and position of the external magnet using the support arm, and thereby the strength and position of the magnetic field, the magnetic positioning device may control the position of the intracavity devices disposed within a body cavity or lumen of a patient. In some variations, a strength and/or position of the external magnet may be used to control a force of a magnetically coupled intracavity device against a body cavity wall or lumen wall using the sensors described in detail herein.

### K. Sensors

The surgery systems described herein may optionally comprise one or more sensors to determine a location of a portion of one or more support arms, external magnetic positioning devices (e.g., external magnets), patient body surfaces (e.g., abdomen, internal cavity wall, breasts), surgery system components (e.g., end effector, intracavity devices, trocar, control console), and operator. For example, an end effector connector may comprise one or more sensors configured to detect a location of a patient body surface and calculate a proximity of the end effector connector relative to the patient such that the controller may ensure that the support arm and/or the end effector connector do not contact the patient. For example, each segment of a support arm may comprise an inductive proximity sensor to calculate a distance between the support arms. As another example, an infrared, radar, or ultrasonic range finder mounted on the support arm and/or external arm may be configured to calculate a distance to the patient. As yet another example, the end effector connector may comprise optical sensors internal and/or external to the support arms configured to visualize the other support arms, operator, input/output device, patient platform, patient, and the like. A controller may be configured to maintain a predetermined distance between the end effector connector and a patient body surface such as a distance of about 1 mm, about 5 mm, or about 10 mm. Thus, a controller may limit a range of motion of the support arm.

As another example, a position of an end effector may be controlled using a force sensor of the end effector connector, such as for an end effector in contact with an internal body cavity wall. A contact force of the end effector with the body cavity wall may be reduced if a force sensor detects that the force exceeds a predetermined threshold. The sensors may comprise one or more of a force sensor (e.g., Hall sensor, load cell, springs), proximity sensor, optical sensor, motion sensor, accelerometer, gyroscope, laser rangefinder, radar, and LIDAR.

### L. Input Device

Generally, an input device 122 of a robotic surgery system 100 may serve as a communication interface between an operator and the surgery system 100. The input device 122 may be configured to receive input data and output data to one or more of the support arm 112, sensor 120, end effector 118, and output device 130. For example, operator control of an input device 122 (e.g., foot controller, joystick, keyboard, touch screen) may be processed by processor 124 and memory 126 for input device 122 to output a control signal to one or more end effectors 118, support arms 120. As another example, images generated by an end effector 118 comprising a visualization device (e.g., endoscope) may be received by input device 122, processed by processor 124 and memory 126, and displayed by the output device 130 (e.g., monitor display). Sensor data from one or more sensors 120 may be received by input device 122 and output visually, audibly, and/or through haptic feedback by one or more output devices 130.

In some variations, a single operator may control one or more components of a surgery system 100 using one or more input devices 122. Some variations of an input device may comprise at least one switch configured to generate a control signal. The input device may be coupled to a support arm and/or disposed on a patient platform or medical cart adjacent to the patient and/or operator. However, the input device may be mounted to any suitable object, such as furniture (e.g., a bed rail), a wall, a ceiling, or may be self-standing. The control signal may include, for example, a movement signal, device switch signal, activation signal, magnetic field strength signal, and other signals. In some variations, the input device may comprise a wired and/or wireless transmitter configured to transmit a control signal to a wired and/or wireless receiver of a controller. A movement signal (e.g., for the control of movement, position, and orientation) may control movement in at least four degrees of freedom of motion, and may include yaw and/or pitch rotation. For example, an input device comprising a touch surface may be configured to detect contact and movement on the touch surface using any of a plurality of touch sensitivity technologies including capacitive, resistive, infrared, optical imaging, dispersive signal, acoustic pulse recognition, and surface acoustic wave technologies. The exemplary input control scheme depicted in FIGS. 7A-7D is discussed in further detail herein.

In some variations, a foot controller may be configured to operate one or more support arms and end effectors of a robotic surgery system described herein. FIGS. 7A and 7D depicts perspective views of an input device 700 of a robotic surgery system. FIG. 7B depicts a side view and FIG. 7C depicts a top view of the input device 700. The input device 700 may comprise a base 710 (e.g., midfoot rest), a set of forefoot switches 720, 722, 724, 726, and a set of hindfoot switches 730, 732. In some variations, the set of switches may comprise a first switch 720, a second switch 722, a third switch 724, a fourth switch 726, a fifth switch 728, a sixth switch 730, and a seventh switch 732. In some variations, an operator may stand on the input device 700 such that at a resting position, neither the forefoot nor the midfoot activates any of the switches 720, 722, 724, 726, 728, 730, 732. Additionally or alternatively, the operator may operate the input device 700 from a sitting position. In some variations, the set of switches 720, 722, 724, 726, 728, 730, 732 may be pressure sensitive.

In some variations, motion of an operator foot may correspond to motion of a support arm. In some variations, a forward motion (e.g., translation) of the foot may activate first switch 720 and correspond to a forward motion (e.g., translation) of the support arm and any end effector coupled thereto. In some variations, a backward motion (e.g., translation) of the foot may activate the sixth switch 730 and may correspond to a backward motion of the support arm.

In some variations, a downward motion of the forefoot (e.g., extension) may activate the second switch 722 and may correspond to a downward motion of the support arm. In some variations, an upward motion of the forefoot (e.g., flexion) may activate the fifth switch 728 and may correspond to an upward motion of the support arm.

In some variations, a leftward motion (e.g., yaw) of the forefoot may activate the third switch 724 and may correspond to a leftward motion of the support arm. In some variations, a rightward motion (e.g., yaw) of the forefoot may activate the fourth switch 726 and may correspond to a rightward motion of the support arm.

In some variations, a downward motion of the hindfoot (e.g., heel down motion) may activate the seventh switch 732 and may correspond to a device switching signal. For example, activating the seventh switch 732 may switch input device 700 control between a first support arm, a second support arm, an end effector, and the like. The downward hindfoot motion is distinct from the other foot motions (forward, back, up, down, left, right), and may thus reduce accidental inputs such as device switches. Thus, a single foot of an operator may be configured to control a robotic surgery system while leaving the operator's visual attention and hands available for other tasks.

In some variations, the input device 700 may comprise additional switches that correspond to additional movements of a support arm (e.g., combination movements such as forward and downward, backwards and leftward).

Additionally or alternatively, the set of switches 720, 722, 724, 726, 730, 732 may be programmed with different functions according to operator preference. In some variations, the set of switches 720, 722, 724, 726, 730, 732 may comprise one or more of a mechanical switch, optical sensor, accelerometer (e.g., 3-axis), gyroscope (e.g., 3-axis), motion sensor, pressure sensor, magnetic sensor, combinations thereof, and the like. In some variations, an operator foot may be releasably coupled (e.g., strapped) to the input device 700.

In variations of an input device comprising at least one switch, a switch may comprise, for example, at least one of a button (e.g., hard key, soft key), touch surface, keyboard, analog stick (e.g., joystick), directional pad, mouse, trackball, jog dial, step switch, rocker switch, pointer device (e.g., stylus), motion sensor, image sensor, and microphone. A motion sensor may receive operator movement data from an optical sensor and classify an operator gesture as a control signal. A microphone may receive audio and recognize an operator voice as a control signal. In variations of a system comprising a plurality of input devices, different input devices may generate different types of signals. For example, some input devices (e.g., button, analog stick, directional pad, and keyboard) may be configured to generate a movement signal while other input devices (e.g., step switch, rocker switch) may be configured to transition a component of the surgery system (e.g., support arm, sensor, intracavity device) between a first configuration and second configuration (e.g., on and off, extended and retracted, open and closed).

In some variations, a single input device may be configured to control a plurality of system components (e.g., intracavity devices, support arms). For example, a touch surface of an input/output device may be configured to control a plurality of external magnetic positioning devices and/or intracavity devices through a set of device selector buttons and device control buttons.

In other variations, a plurality of input devices may be configured to control a single component of the surgery system (e.g., intracavity device) to enhance operator flexibility. For example, an operator may choose to control a support arm using combinations of a joystick, directional pad, soft keys, voice commands, and the like.

In still other variations, each input device of a surgery system may be associated with a corresponding component of the surgery system. Some non-limiting examples include: a joystick may be configured to control movement of a support arm; a touch screen may be configured to pan, tilt, and/or zoom a visualization device; a jog dial may be configured to control the jaw positions of a grasper; and a step switch may be configured to release a delivery device from an intracavity device.

In variations of the input device comprising one or more buttons, button presses of varying duration may execute different functions. For example, a lumen output level of a light source may be configured to increase with a longer button press. Conversely, a shorter duration button press may correspond to a different function such as deactivating the light source.

In some variations, a surgery system may comprise a plurality of input devices provided in separate housings, where for example a first input device may be handheld and/or portable while a second input device may be stationary. In some variations, a first input device may comprise a tablet including a touch screen display and a second input device may comprise a step switch or foot pedal. The step switch may in some variations be a safety switch that must be engaged at the same time as contact with the touch screen before a control signal is transmitted to the surgery system. Output of a control signal upon simultaneous engagement of a first input device and second input device may confirm that operator input to the first input device is intentional.

### M. Processor

A surgery system 100, as depicted in FIG. 1, may comprise a processor 124 and a machine-readable memory 126 (e.g., controller) in communication with one or more support arms 112 and/or end effectors 118. The processor 124 may be connected to the support arms 112 and/or end effectors 118 by wired or wireless communication channels. The processor 124 may be located in the same or different room as the patient. In some variations, the processor 124 may be coupled to a patient platform or disposed on a medical cart adjacent to the patient and/or operator. The processor 124 may be configured to control one or more components of the system 100, such as an end effector 118 that may visualize a body cavity or lumen, grasp tissue, retract tissue, hold and/or drive a needle, and the like. In some variations, the processor 124 may be configured to coordinate movement and orientation of end effectors 118 within a body cavity or lumen through corresponding movement and control of the support arm 112.

The processor 124 may be implemented consistent with numerous general purpose or special purpose computing systems or configurations. Various exemplary computing systems, environments, and/or configurations that may be suitable for use with the systems and devices disclosed herein may include, but are not limited to software or other components within or embodied on personal computing devices, network appliances, servers or server computing devices such as routing/connectivity components, portable (e.g., hand-held) or laptop devices, multiprocessor systems, microprocessor-based systems, and distributed computing networks.

Examples of portable computing devices include smartphones, personal digital assistants (PDAs), cell phones, tablet PCs, phablets (personal computing devices that are larger than a smartphone, but smaller than a tablet), wearable computers taking the form of smartwatches, portable music devices, and the like, and portable or wearable augmented reality devices that interface with an operator's environment through sensors and may use head-mounted displays for visualization, eye gaze tracking, and user input.

The processor 124 may incorporate data received from memory 126 and operator input to control one or more support arms 112 and end effectors 118. The memory 114 may further store instructions to cause the processor 124 to execute modules, processes, and/or functions associated with the system 100. The processor 124 may be any suitable processing device configured to run and/or execute a set of instructions or code and may comprise one or more data processors, image processors, graphics processing units, physics processing units, digital signal processors, and/or central processing units. The processor 124 may be, for example, a general purpose processor, a Field Programmable Gate Array (FPGA), an Application Specific Integrated Circuit (ASIC), configured to execute application processes and/or other modules, processes, and/or functions associated with the system and/or a network associated therewith. The underlying device technologies may be provided in a variety of component types such as metal-oxide semiconductor field-effect transistor (MOSFET) technologies like complementary metal-oxide semiconductor (CMOS), bipolar technologies like emitter-coupled logic (ECL), polymer technologies (e.g., silicon-conjugated polymer and metal-conjugated polymer-metal structures), mixed analog and digital, combinations thereof, and the like.

### N. Memory

Some variations of memory 126 described herein relate to a computer storage product with a non-transitory computer-readable medium (also may be referred to as a non-transitory processor-readable medium) having instructions or computer code thereon for performing various computer-implemented operations. The computer-readable medium (or processor-readable medium) is non-transitory in the sense that it does not include transitory propagating signals per se (e.g., a propagating electromagnetic wave carrying information on a transmission medium such as air or a cable). The media and computer code (also may be referred to as code or algorithm) may be those designed and constructed for a specific purpose or purposes. Examples of non-transitory computer-readable media include, but are not limited to, magnetic storage media such as hard disks, floppy disks, and magnetic tape; optical storage media such as Compact Disc/Digital Video Discs (CD/DVDs), Compact Disc-Read Only Memories (CD-ROMs), and holographic devices; magneto-optical storage media such as optical discs; solid state storage devices such as a solid state drive (SSD) and a solid state hybrid drive (SSHD); carrier wave signal processing modules; and hardware devices that are specially configured to store and execute program code such as Application-Specific Integrated Circuits (ASICs), Programmable Logic Devices (PLDs), Read-Only Memory (ROM), and Random-Access Memory (RAM) devices. Other variations described herein relate to a computer program product, which may include, for example, the instructions and/or computer code disclosed herein.

The systems, devices, and/or methods described herein may be performed by software (executed on hardware), hardware, or a combination thereof. Software modules (executed on hardware) may be expressed in a variety of software languages (e.g., computer code), including C, C++, Java®, Python, Ruby, Visual Basic®, and/or other object-oriented, procedural, or other programming language and development tools. Examples of computer code include, but are not limited to, micro-code or micro-instructions, machine instructions, such as produced by a compiler, code used to produce a web service, and files containing higher-level instructions that are executed by a computer using an interpreter. Additional examples of computer code include, but are not limited to, control signals, encrypted code, and compressed code.

### O. Communication Device

In some variations, surgery systems 100 described herein may communicate with networks and computer systems through a communication device 128. In some variations, the surgery system 100 may be in communication with other devices via one or more wired and/or wireless networks. A wireless network may refer to any type of digital network that is not connected by cables of any kind. Examples of wireless communication in a wireless network include, but are not limited to cellular, radio, satellite, and microwave communication. However, a wireless network may connect to a wired network in order to interface with the Internet, other carrier voice and data networks, business networks, and personal networks. A wired network is typically carried over copper twisted pair, coaxial cable and/or fiber optic cables. There are many different types of wired networks including wide area networks (WAN), metropolitan area networks (MAN), local area networks (LAN), Internet area networks (IAN), campus area networks (CAN), global area networks (GAN), like the Internet, and virtual private networks (VPN). Hereinafter, network refers to any combination of wireless, wired, public and private data networks that are typically interconnected through the Internet, to provide a unified networking and information access system.

Cellular communication may encompass technologies such as GSM, PCS, CDMA or GPRS, W-CDMA, EDGE or CDMA2000, LTE, WiMAX, and 5G networking standards. Some wireless network deployments combine networks from multiple cellular networks or use a mix of cellular, Wi-Fi, and satellite communication. In some variations, the network interface 116 may comprise a radiofrequency receiver, transmitter, and/or optical (e.g., infrared) receiver and transmitter. The communication device 128 may communicate by wires and/or wirelessly with one or more of the support arm 112, end effector 118, sensor 120, input device 122, output device 130, network, database, server, combinations thereof, and the like.

### P. Output Device

An output device 130 of a surgery system 100 may be configured to output data corresponding to a surgery system, and may comprise one or more of a display device, audio device, and haptic device. The output device may be coupled to a patient platform and/or disposed on a medical cart adjacent to the patient and/or operator. In other variations, the output device may be mounted to any suitable object, such as furniture (e.g., a bed rail), a wall, a ceiling, and may be self-standing.

A display device may allow an operator to view images of one or more end effectors, support arms, body cavities, and tissue. For example, an end effector comprising a visualization device (e.g., camera, optical sensor) located in a body cavity or lumen of a patient may be configured to image an internal view of the body cavity or lumen and/or intracavity devices. An external visualization device may be configured to image an external view of the patient and one or more external magnetic positioning devices. Accordingly, the display device may output one or both of internal and external images of the patient and system components. In some variations, an output device may comprise a display device including at least one of a light emitting diode (LED), liquid crystal display (LCD), electroluminescent display (ELD), plasma display panel (PDP), thin film transistor (TFT), organic light emitting diodes (OLED), electronic paper/e-ink display, laser display, and/or holographic display.

An audio device may audibly output patient data, sensor data, system data, alarms and/or warnings. For example, the audio device may output an audible warning when monitored patient data (e.g., blood pressure) falls outside a predetermined range or when a malfunction in a support arm is detected. As another example, audio may be output when operator input is overridden by the surgery system to prevent potential harm to the patient and/or surgery system (e.g., collision of support arms with each other, excessive force of the intracavity device against a patient cavity wall). In some variations, an audio device may comprise at least one of a speaker, piezoelectric audio device, magnetostrictive speaker, and/or digital speaker. In some variations, an operator may communicate to other users using the audio device and a communication channel. For example, the operator may form an audio communication channel (e.g., VoIP call) with a remote operator and/or observer.

A haptic device may be incorporated into one or more of the input and output devices to provide additional sensory output (e.g., force feedback) to the operator. For example, a haptic device may generate a tactile response (e.g., vibration) to confirm operator input to an input device (e.g., touch surface). Haptic feedback may in some variations simulate a resistance encountered by an intracavity device within a body cavity or lumen (e.g., magnetic field and tissue resistance). Additionally or alternatively, haptic feedback may notify that an operator input is overridden by the surgery system to prevent potential harm to the patient and/or system (e.g., collision of support arms with each other). Operator interaction with a user interface utilizing an input and output device is discussed in more detail herein.

### II. Methods

Also described here are methods for treating a patient using the surgery systems described herein. A single operator may operate a surgery system comprising a plurality of intracavity devices without requiring assistance from another operator to operate the surgery system. Generally, the methods described here comprise performing magnetic laparoscopic robotic surgery including coupling and decoupling an end effector to a robotic arm within a sterile field using a single operator. The robotic surgery methods may thus enable single operator end effector exchange and robotic arm control for laparoscopic procedures. Moreover, the robotic surgery system may ensure patient safety by locating and monitoring a position and orientation of an end effector relative to a trocar, monitoring one or more of a position of an external end effector relative to the patient, and monitoring of an end effector attachment status. Since multiple components of the surgery system may be electronically or manually controllable via a single operator, the methods described here may allow a single operator to perform a surgical procedure, even when that surgical procedure involves a number of tools. This may have numerous benefits, such as reducing the cost of surgery.

### A. Single Operator Robotic Surgery

Single operator robotic surgery may include a single operator end effector exchange that does not affect a sterile field. For example, FIG. 8 is flowchart that generally describes a method of performing robotic surgery 800 such as performing magnetic laparoscopic robotic surgery using any of the systems and devices described herein. The method 800 may include magnetically coupling an end effector to a support arm (e.g., robotic arm) within a sterile field using an end effector connector 802. The end effector may be controlled by the support arm and/or the operator within a body cavity of a patient 804. For example, an end effector such as a visualization device (e.g., endoscope) may be advanced into the body cavity by the support arm through a trocar to provide visualization for a surgical procedure being performed. The support arm may moveably suspend the end effector coupled thereto within the body cavity.

Additionally or alternatively, the end effector may be controlled externally of the patient. For example, a magnetic positioning device may be magnetically coupled to the support arm and be moved above an external surface of the patient to position an intracavity device (e.g., grasper) located within the body cavity. In some variations, a portion of the end effector may be controlled within the body cavity of the patient and another portion of the end effector may be controlled externally of the patient. In some variations, surgery may be performed using the end effector 806. In some variations, the end effector may be decoupled from the support arm 808 within and while maintaining the sterile field using the end effector connector. For example, a first end effector (e.g., delivery device) may be withdrawn from the body cavity and the end effector connector may be actuated to physically decouple the end effector from the support arm. Another end effector may be coupled to the support arm using the end effector connector 810. For example, a second end effector (e.g., retractor) may be physically coupled to the support arm by the single operator to perform another step of the surgery.

One or more of the steps of method 800 (e.g., coupling 802, controlling 804, performing 806, decoupling 808, coupling 810) may be performed by a single operator without the aid of another person. In some variations, one or more of the steps may be performed using a single hand of the single operator. For example, controlling the end effector 804 is capable of being performed by a single hand of the single operator. Coupling and decoupling the end effector 802, 808, 810 is capable of being performed by two hands of the single operator. Coupling, controlling, and decoupling the end effector may be performed while maintaining the sterile field.

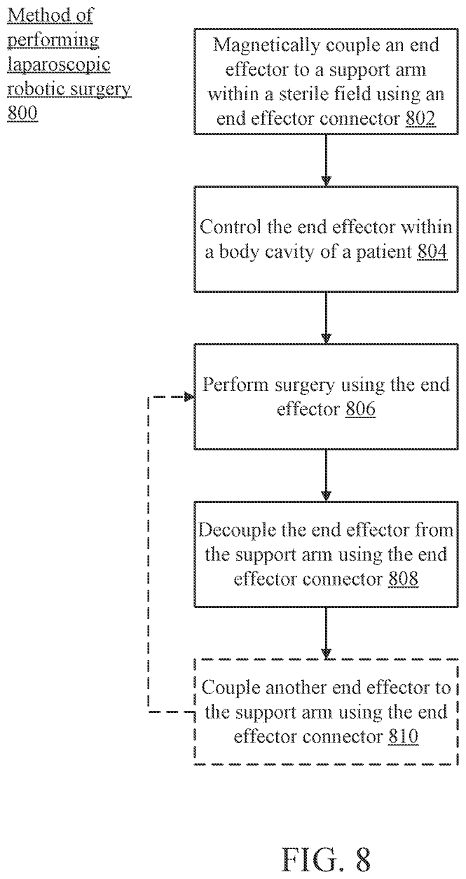

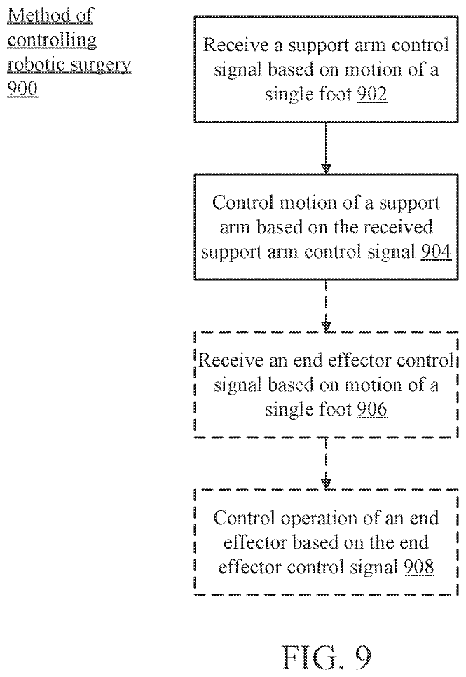

In some variations, single operator robotic surgery may include providing control inputs to a robotic surgery system using a single foot of the operator, thereby freeing the visual attention and hands of the operator for other aspects of a procedure. FIG. 9 is an illustrative method of controlling robotic surgery 900 such as performing magnetic laparoscopic robotic surgery using any of the systems and devices described herein. The method 900 may include receiving a support arm control signal based on motion of a single foot of an operator 902. In some variations, a support arm control signal may be received based on motion of a single foot of an operator. In some variations, the operator may be standing during the motion of the single foot. In some variations, a support arm control signal may be received based on motion of a single foot 904.

Motion of a support arm may be controlled with at least three degrees of freedom based on the received robotic arm control signal 906. In some variations, the support arm control signal may comprise a translation motion of the support arm corresponding to a translation motion of the single foot. In some variations, the support arm control signal may comprise a lateral motion of the support arm corresponding to a lateral motion or a yaw motion of the single foot. In some variations, the support arm control signal may comprise a downward motion of the support arm corresponding to a flexion motion of the single foot. In some variations, the support arm control signal may comprise a support arm switch command corresponding to a heel movement of the single foot.

In some variations, the method 900 may optionally include receiving an end effector control signal based on motion of a single foot of an operator 906. In some variations, an operation of an end effector may be controlled based on the end effector control signal 908.

### B. Decoupling an End Effector

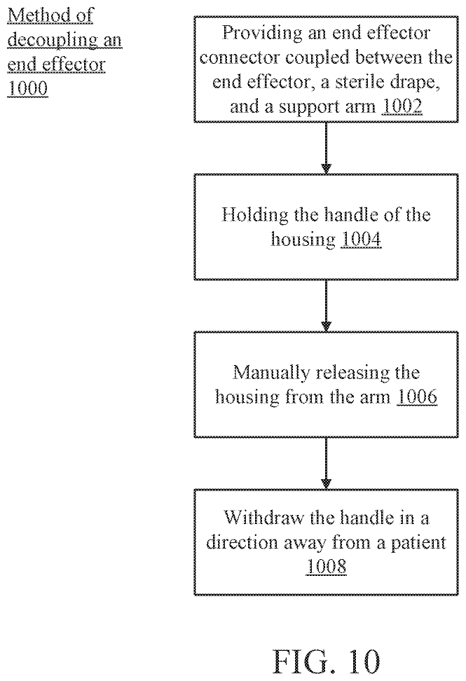

A method of decoupling an end effector from a support arm may be performed using the devices described herein. For example, FIG. 10 is an illustrative method of decoupling an end effector from a robotic surgery system 1000. The method 1000 may include providing an end effector connector coupled between the end effector, a sterile drape, and a robot (e.g., support arm) 1002. In some variations, the end effector connector may comprise any of the end effectors described in detail herein. For example, the end effector connector may comprise an arm releasably coupled to a housing. The housing may comprise a handle. In some variations, the arm may comprise a magnetic portion configured to magnetically couple to the support arm.

In some variations, a handle of the housing of the end effector connector may be held by an operator 1004. For example, the handle may be held by a single hand of an operator. Although the end effector may be held and moved automatically using a robot, the handle may improve the ergonomics of using the end effector when manual control is desired by an operator. For example, the operator may quickly and easily perform fine manual adjustment of the position and/or orientation of the end effector by manipulating the handle of the housing of the end effector.

In some variations, the housing may be manually released from the arm 1006. In some variations, manually releasing the housing from the arm may include withdrawing the released end effector in a direction away from a patient (e.g., so as to not damage the patient as the end effector is being withdrawn from a body cavity). Additionally or alternatively, holding the handle and manually releasing the housing may be simultaneously performed by a single hand. Thus, decoupling an end effector from a robot (e.g., robot arm, support arm) may be quickly and safely performed by a single operator since the end effector is withdrawn after releasing the housing from the arm in a direction away from the patient without damaging tissue.

### C. Coupling an End Effector

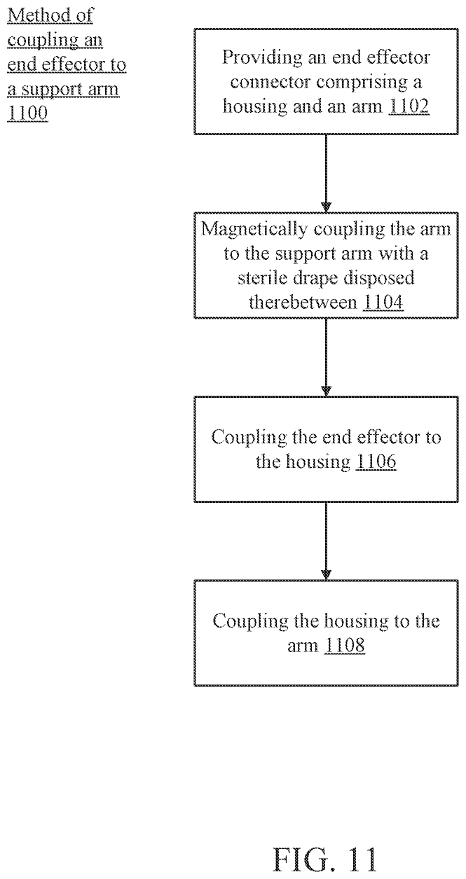

A method of coupling an end effector to a support arm may be performed using the devices described herein. For example, FIG. 11 is an illustrative method of coupling an end effector to a support arm (e.g., robot, robotic arm) 1100. The method 1100 may include providing an end effector connector comprising a housing and an arm 1102. The arm may be configured to be releasably coupled to the housing. In some variations, the arm of the end effector connector may be magnetically coupled to the support arm with a sterile drape disposed therebetween 1104. In this manner, a sterile field may be set up prior to coupling an end effector to the support arm. In some variations, the end effector may be coupled to the housing of the end effector connector 1106. For example, each end effector to be held by a support arm during a procedure may be coupled to a respective housing, thereby facilitating efficient end effector exchange.

In some variations, the housing of the end effector connector (having the end effector coupled thereto) may be coupled to the arm of the end effector connector 1108. For example, a proximal end of the housing may be brought into mechanical alignment with a distal end of the arm using a housing release mechanism as described herein. In some variations, coupling the housing to the arm may be performed by a single hand of an operator. For example, the single hand of the operator may hold a handle of the housing having the end effector coupled thereto. The operator may mechanically couple the housing to the arm via the housing release mechanism to thereby couple an end effector to a support arm via the end effector connector.

### D. End Effector Registration and Control

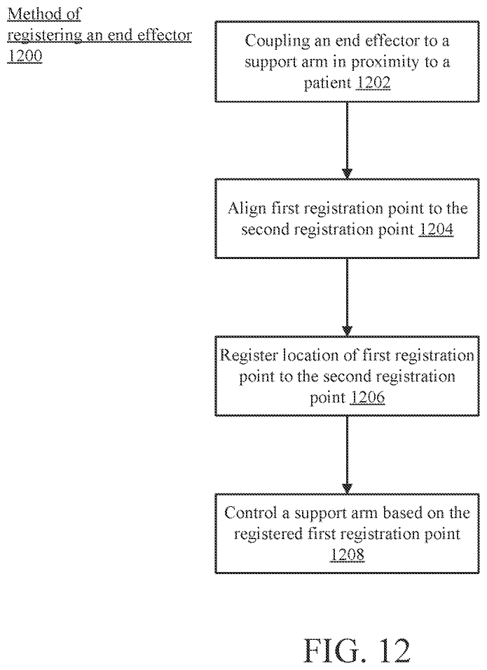

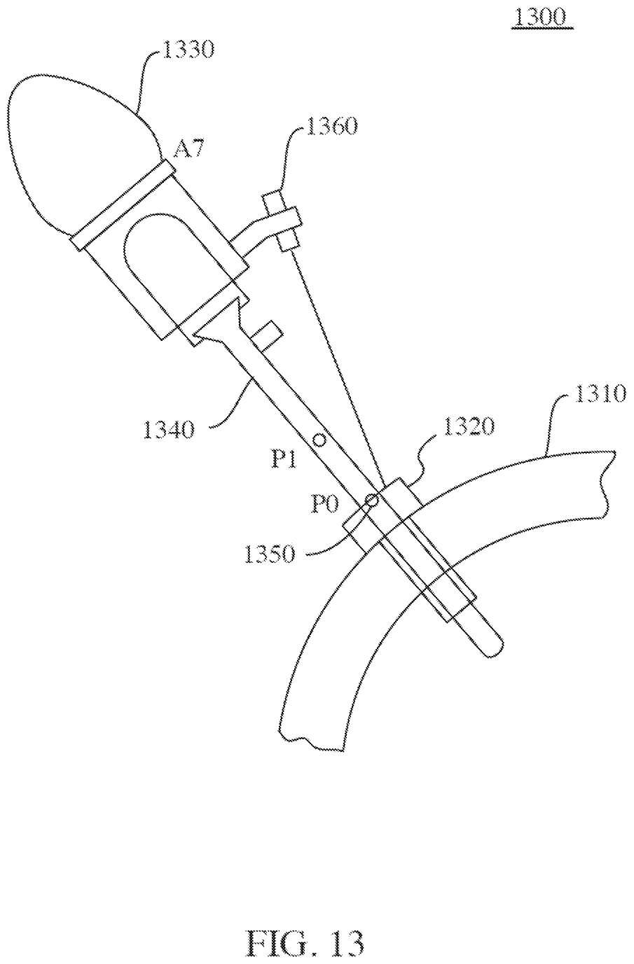

A method of registering an end effector within three-dimensional space may facilitate robotic control of an end effector and ensure the safety of the patient. FIG. 12 is an illustrative method of registering an end effector 1200. The method 1200 may include coupling an end effector to a support arm (e.g., robot, robotic arm) of a robotic surgery system in proximity to a patient 1202 such as described herein. For example, FIG. 13 depicts a procedure 1300 where a trocar 1320 protrudes through a body wall 1310 of a patient. An end effector 1340 (e.g., endoscope) may be coupled to a robotic arm 1330. The end effector may comprise a first registration point and the patient may comprise a second registration point. In some variations, the first registration point may intersect a longitudinal axis of the end effector. For example, the end effector may comprise a first registration point 1350 (e.g., virtual tool center) provided along a longitudinal axis of the end effector 1340. In some variations, the second registration point may correspond to an access site of the patient. For example, the second registration point may correspond to an inlet of a trocar 1320 coupled to the patient.

The first registration point of the end effector may be aligned to the second registration point 1204 (e.g., access site, incision site, port, trocar). Aligning the first registration point to the second registration point may comprise overlapping the first registration point to the second registration point. For example, a distal portion of end effector 1340 may be advanced partway into a patient through trocar 1320. In some variations, the first registration point 1350 may correspond to a location where the trocar 1320 intersects an incision in the patient. In some variations, the second registration point may correspond to a muscle layer of an abdominal wall of the patient. The operator may manually manipulate (e.g., guide) the end effector 1340 and robotic arm 1330 to align the first registration point 1350 of the end effector 1340 to, for example, an inlet of the trocar 1320. Additionally or alternatively, the support arm may be controlled to align the first and second registration points.

In some variations, an illumination source 1360 (e.g., laser) may be used to virtually mark and highlight the first registration point 1350 on the end effector 1340 in a sterile, non-contact manner. This enables end effector registration even if the end effector 1340 is absent a permanent registration point. In some variations, a visual indicator of the first registration point of the end effector may be generated. In some variations, the visual indicator may comprise illumination directed at the end effector.

A location of the first registration point aligned to the second registration point may be registered 1206. For example, the location of the first registration point may be registered in a three-dimensional coordinate system of the robotic surgery system. One or more of a tool center, trocar location, support arm location and/or orientation, end effector location and/or orientation may be stored in memory and serve as a three-dimensional reference point for robotic arm control. Further movement and/or control of the robotic surgery system may use the end effector registration point as a point of reference. Thus, a single operator may efficiently register the end effector 1340. In some variations, each end effector used in a procedure may be registered for each access site (e.g., port) of a patient.

The support arm (and end effector coupled thereto) may be controlled based on the registered first registration point 1208. In some variations, the registered first registration point may correspond to a pivot point. The robotic surgery system may be configured to pivot the end effector about the pivot point without applying external force or displacement to the patient in order to minimize tissue damage to an access site (e.g., patient incision), thereby reducing healing time and pain.

In some variations, controlling the support arm may comprise one or more of pitching, yawing, rolling, and translating the end effector within a conical range of motion comprising a vertex at the pivot point. The pivot point (e.g., trocar point) may correspond to the point where the trocar intersects one of the incisions of the patient (e.g., an umbilical incision).

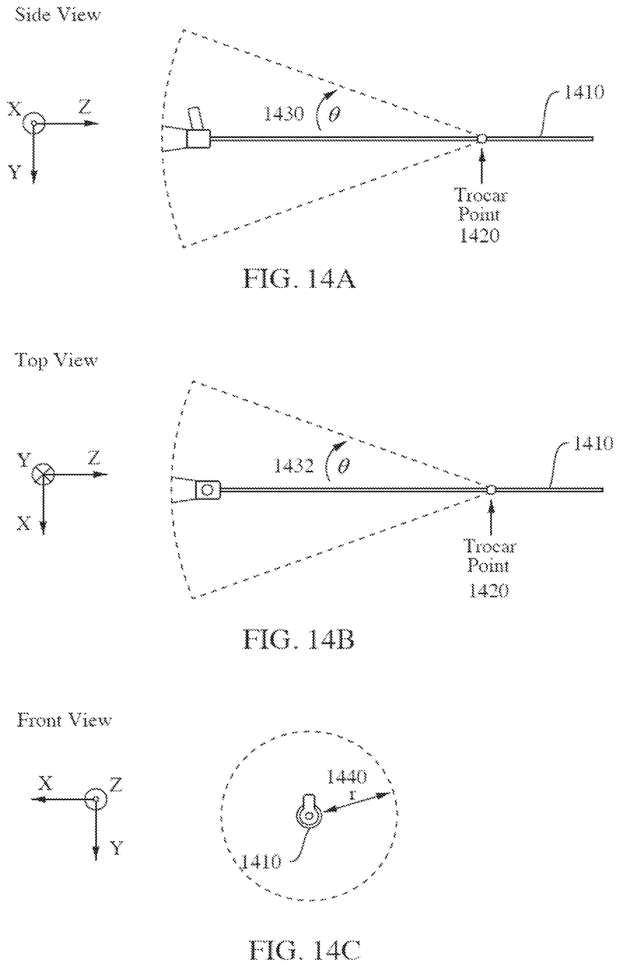

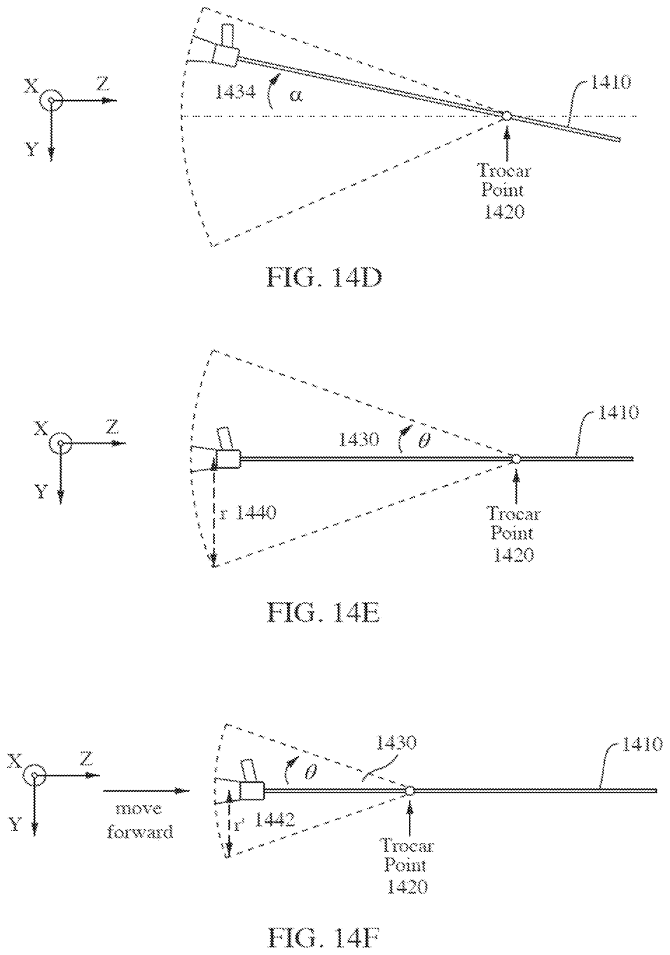

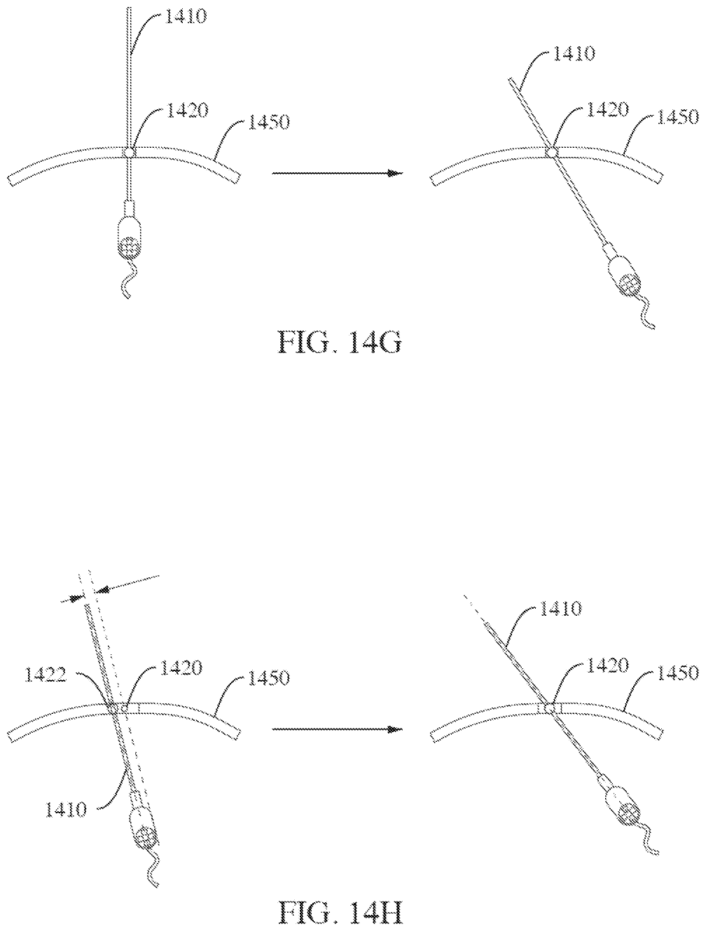

For example, an operator may input a control input to an input device to control the support arm and end effector. Additionally or alternatively, the operator may manually manipulate the end effector using, for example, a housing handle of an end effector connector. In some variations, controlling the support arm may comprise maintaining an intersection of the end effector to the pivot point. In some variations, an operator may input a command to rotate (e.g., pivot) the end effector about the registration point (e.g., pivot point) to control the orientation and translation of the end effector. For example, FIGS. 14A and 14B are schematic side and plan views of an end effector 1410 range of motion end effector about a registered pivot point 1420 (e.g., trocar point) that forms a cone. The end effector 1410 may be rotated up to an angle θ 1430 relative to the pivot point to pitch (e.g., FIG. 14A) and yaw (e.g., FIG. 14B) the end effector 1410.

In some variations, an operator may input one or more of an up, down, left, and right movement command using an input device (e.g., input device 700) to move an end effector about the pivot point 1420 in a corresponding direction while maintaining intersection of the end effector 1410 through the pivot point 1420. For example, up and down input movement may correspond to respective pitch up and pitch down movement at an angle a as shown in FIG. 14D. Similarly, left and right input movement may correspond to respective yaw left and yaw right an angle β of the end effector. A combination of pitch angle α and yaw angle β movement may be constrained by the cone surface defined by the maximum cone angle θ and represented by the equation: α²+β²≤θ². In some variations, the conical range of motion may have a maximum cone angle θ of up to about 50 degrees. This may facilitate movement within a body cavity (e.g., abdomen) of the patient.

As shown in FIG. 14C, a proximal portion of the end effector 1410 may have a radius r 1440 formed by the cone having a vertex at the pivot point 1420. In some variations, end effector 1440 translation in a forward and backward direction may change the radius r 1440. For example, forward motion of the end effector 1410 in FIG. 14F corresponds to a radius r′ 1442 smaller than radius r 1440. While the trocar point 1420 is fixed in space, the point along the end effector 1410 which intersects the trocar point 1420 changes. That is, translation of the end effector 1410 changes the relative pivot point of the end effector 1410.

In some variations, reregistration may be required when the pivot point changes. For example, the pivot point should be reregistered if the end effector is advanced into the patient through a different access site (e.g., different trocar) or if a coordinate system of the system has changed (e.g., due to movement of the base). In some variations, the first registration point may be reregistered to the second registration point when one or more of the patient moves relative to a patient platform, the patient platform moves, a base of a support arm is moved, the pivot point is moved, or when the incision point otherwise suffers a significant displacement.

In some variations, the registered first registration point may function as a reference default position of the end effector. For example, as a support arm and/or operator moves the end effector 1410 as shown in FIG. 14G, the robotic surgery system may continuously monitor the position and/or orientation of the end effector based on the registered first registration point 1420. The operator may optionally hand guide the end effector. In some variations, the operator may hand guide the end effector out of the patient 1450 (e.g., for end effector cleaning) and may re-enter the patient 1450 while maintaining the registered first registration point. As shown in FIG. 14H, an end effector 1410 misaligned 1422 within an access site of the patient 1450 due to hand guided movements may be automatically aligned by the support arm of the robotic surgery system back to the registered first registration point 1420.

### E. End Effector Monitoring

A method of monitoring an end effector may ensure the safety of the patient by limiting pressure applied by an end effector to the patient such as a magnetic positioning device. Some end effectors such as a magnetic positioning device may be coupled to a support arm and disposed externally of a patient during a surgical procedure. The patient may be injured if either the patient or the magnetic positioning device inadvertently moves toward each other such that a traumatic force is applied to the patient. For example, FIG. 16 illustrates a patient 1610 disposed on a patient platform 1620. An end effector 1640 (e.g., magnetic positioning device) may be coupled to a support arm (e.g., robotic arm) 1630 and held in proximity to the patient 1610. In some variations, the end effector 1640 may be positioned at a predetermined distance away from the patient 1610. Additionally or alternatively, the end effector 1640 may be in contact with the patient 1610 (e.g., placed atraumatically in contact with the skin of the patient 1610). However, the patient 1610 may become injured if, for example, the patient platform 1620 tilts upward toward the end effector 1640 while the end effector 1640 remains in a stationary position or if the end effector 1640 moves toward the patient 1610 while the patient platform 1620 remains in a stationary position.

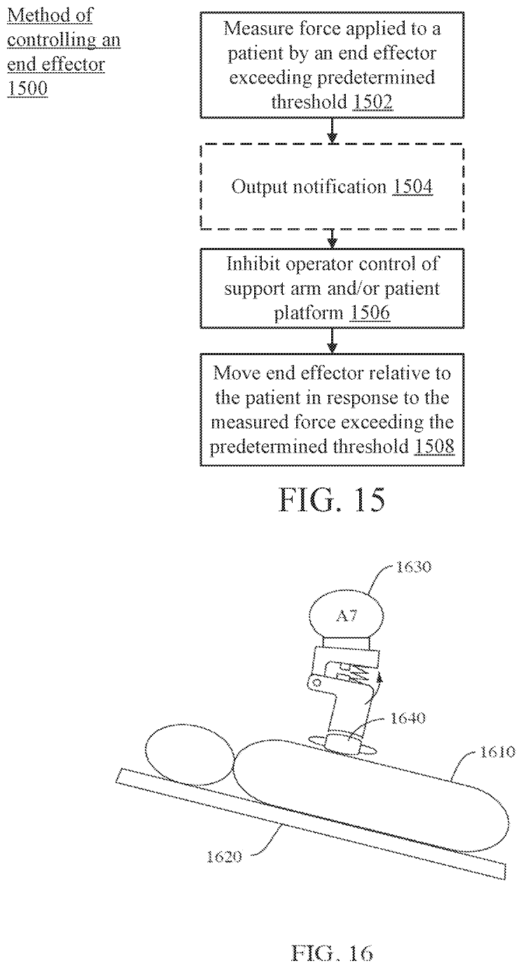

FIG. 15 is an illustrative method of controlling (e.g., monitoring) an end effector 1500. The method 1500 may include measuring a force applied to a patient by an end effector coupled to a support arm 1502. The measure force may be compared to a predetermined threshold (e.g., safety threshold). For example, the support arm 1630 and/or end effector 1640 may comprise one or more force sensors configured to measure a force applied against the end effector 1640. Optionally, a notification may be outputted (e.g., to an operator) in response to the measured force exceeding the predetermined threshold 1504. For example, one or more of an audio alarm, visual output, and haptic feedback may be output to warn the operator of potential and/or imminent injury. In some variations, operator control of one or more of the support arm and patient platform may be inhibited in response to the measured force exceeding the predetermined threshold 1506. For example, an operator may provide a control signal to move the end effector 1640 toward the patient 1610 in a manner that would exceed the predetermined force threshold. However, such a motion may be inhibited by the system to reduce patient injury.

The end effector may be moved (e.g., translated, rotated) relative to the patient in response to the measured force exceeding the predetermined threshold 1508. For example, the end effector 1640 may be automatically moved away from the patient (e.g., moved upward) by a predetermined amount in order for the end effector 1640 to separate (e.g., breakaway) from the patient. For example, the end effector 1640 may be moved by the support arm 1630 upward by a first distance (e.g., about 2 cm) in response to operator input to move the end effector 1640 downward in a manner that exceeds a predetermined force threshold. Furthermore, if the measured force continues to exceed the predetermined threshold, then the end effector 1640 may be moved upward by a second distance (e.g., 10 cm). This may occur if, for example, the patient platform 1620 is accidentally tilted upward while the end effector 1640 remains in a stationary position. A larger second distance relative to the first distance may reduce patient injury.

In some variations, moving the end effector relative to the patient may comprise moving the support arm 1630 coupled to the end effector 1640. In some variations, a patient platform 1620 may be controlled in response to the measured force exceeding the predetermined threshold. For example, the patient platform may be moved away (e.g., downward) from the end effector 1640 to separate the patient 1610 from the end effector 1640. In some variations, the end effector 1640 may comprise a magnetic positioning device configured to control an intracavity device (not shown) disposed within the patient 1610.

Additionally or alternatively, a method of controlling an end effector may include determining a distance between a patient and an end effector coupled to a support arm using a tracking system comprising one or more of an optical sensor, a patient fiducial coupled to the patient, and an end effector fiducial coupled to the end effector. The tracking system may be configured to monitor the relative positions of the patient and end effector based on the patient monitored positions of the patient fiducial and end effector fiducial. The end effector may be moved relative to the patient based on the determined distance to maintain a predetermined distance between the patient and the end effector. In some variations, moving the end effector 1640 relative to the patient 1610 may comprise maintaining at least a predetermined distance between the end effector 1640 and the patient 1610.

### F. End Effector Decoupling Confirmation

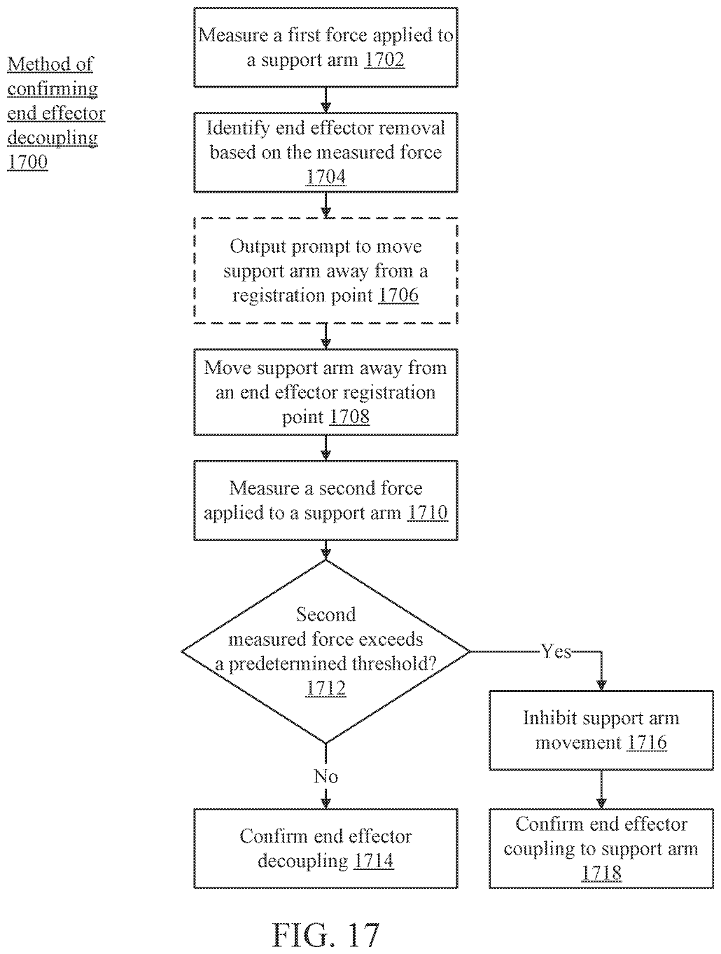

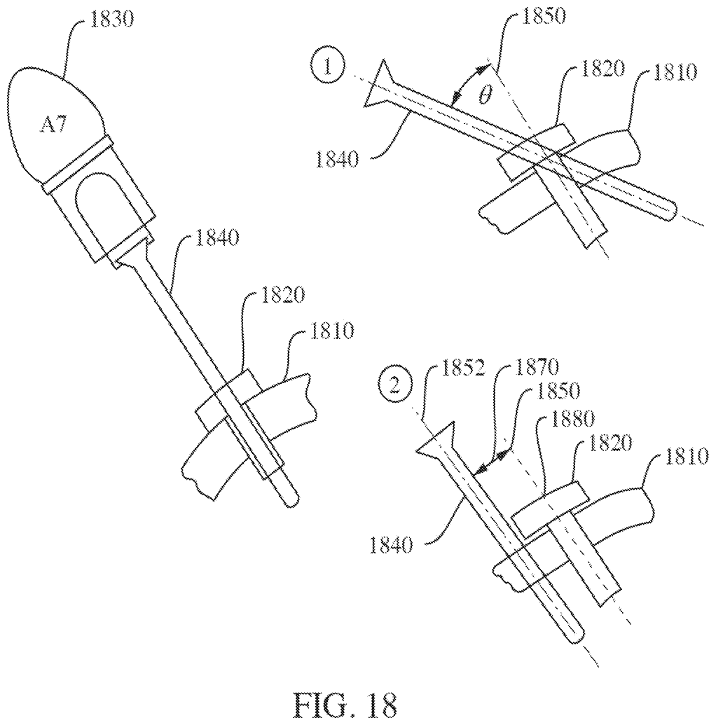

A method of confirming end effector decoupling may ensure the safety of the patient by checking against operator error. FIG. 17 depicts a flowchart representation of end effector decoupling confirmation and FIG. 18 is a corresponding schematic diagram. FIG. 18 is a cross-sectional schematic diagram of a patient body wall 1810, trocar 1820, support arm 1830 (e.g., robotic arm), and end effector 1840. In some variations, a first force applied to a support arm may be measured 1702. End effector removal may be determined based on the measured force exceeding a first predetermined threshold 1704. For example, a brief and intense force measured by a force sensor (e.g., of the support arm, end effector connector, or end effector) may correspond to the first force applied by the operator to decouple an end effector from the support arm 1830. Optionally, an operator may be prompted to move the support arm away from a predetermined registration point 1706 in order to confirm that the end effector has been decoupled from the support arm 1830. The support arm may be moved away from an end effector registration point 1708 while concurrently measuring a second force applied to the support arm 1710. If no resistance is measured 1712—No (e.g., by a force sensor), then end effector decoupling may be confirmed 1714. In some variations, an end effector decoupling signal may be generated. If the second measured force exceeds a second predetermined threshold 1712—Yes, then movement of the support arm may be inhibited 1716 in order to prevent damage to tissue. End effector coupling to the support arm may be confirmed 1718. In some variations, an end effector coupling signal may be generated.

For example, rotating the support arm 1830 by a predetermined angle 0 without meeting resistance from trocar 1820 and body wall 1810 effectively confirms that the end effector 1840 has been decoupled from the support arm 1830. Similarly, moving the support arm 1830 a predetermined distance 1870 away from a registration axis 1850 towards a parallel axis 1852 effectively confirms that the end effector 1840 has been decoupled from the support arm 1830. Otherwise, the end effector 1840 would meet the resistance of one or more of the trocar 1820 and body wall 1810 which would be measured by a force sensor. In some variations, the registration axis 1850 may correspond to a longitudinal axis of the trocar 1820 intersecting a registration point 1880.

Although the foregoing variations have, for the purposes of clarity and understanding, been described in some detail by illustration and example, it will be apparent that certain changes and modifications may be practiced, and are intended to fall within the scope of the appended claims. Additionally, it should be understood that the components and characteristics of the systems and devices described herein may be used in any combination. The description of certain elements or characteristics with respect to a specific figure are not intended to be limiting or nor should they be interpreted to suggest that the element cannot be used in combination with any of the other described elements. For all of the variations described herein, the steps of the methods may not be performed sequentially. Some steps are optional such that every step of the methods may not be performed.

1. An end effector connector, comprising:
a housing configured to receive an end effector; and
an arm configured to releasably couple the housing to a robot, the arm comprising:
a magnetic portion; and
a housing release mechanism configured to manually release the housing from the arm.

2. The end effector connector of claim 1, wherein the magnetic portion is coupled to a proximal portion of the arm.

3. The end effector connector of claim 1, wherein the magnetic portion is configured to magnetically couple the arm to the robot through a sterile drape.

4. (canceled)

5. The end effector connector of claim 1, wherein a first side of the magnetic portion is configured to mechanically and magnetically attach to a flange of the robot.

6. (canceled)

7. The end effector connector of claim 1, wherein the magnetic portion comprises a robot engagement feature configured to reduce a radial shear force.

8.-9. (canceled)

10. The end effector connector of claim 1, wherein the magnetic portion comprises a rotational alignment feature.

11.-12. (canceled)

13. The end effector connector of claim 1, wherein the magnetic portion comprises a magnetic release mechanism configured to manually release the arm from the robot.

14.-16. (canceled)

17. The end effector connector of claim 1, wherein the magnetic portion defines a first longitudinal axis and the housing defines a second longitudinal axis, wherein the first longitudinal axis and the second longitudinal axis are non-parallel.

18.-19. (canceled)

20. The end effector connector of claim 17, wherein the end effector received in the housing defines a third longitudinal axis parallel to the second longitudinal axis.

21. The end effector connector of claim 20, wherein a second angle between the first longitudinal axis and the third longitudinal axis is up to about 40 degrees.

22.-23. (canceled)

24. The end effector connector of claim 1, wherein the arm comprises an arm handle.

25. (canceled)

26. The end effector connector of claim 24, wherein the magnetic portion defines a first longitudinal axis and the arm handle defines a fourth longitudinal axis, wherein the first longitudinal axis and the fourth longitudinal axis are non-parallel.

27. The end effector connector of claim 26, wherein a third angle between the first longitudinal axis and the fourth longitudinal axis is up to about 75 degrees.

28.-33. (canceled)

34. The end effector connector of claim 1, wherein the housing release mechanism comprises a first portion coupled to the arm and a second portion coupled to the housing.

35.-37. (canceled)

38. The end effector connector of claim 34, wherein the housing release mechanism comprises a rotational alignment feature configured to inhibit rotation of the first portion relative to the second portion.

39.-40. (canceled)

41. The end effector connector of claim 1, wherein the housing release mechanism comprises a switch configured to release the housing from the arm.

42.-48. (canceled)

49. The end effector connector of claim 1, wherein a distal portion of the housing is configured to receive the end effector.

50. The end effector connector of claim 1, wherein a proximal portion of the housing is coupled to a lateral sidewall of the housing release mechanism.

51.-55. (canceled)

56. The end effector connector of claim 1, wherein the housing comprises a housing handle, the housing handle defines a handle longitudinal axis and a handle lateral axis, and the housing comprises a first stiffness along the handle longitudinal axis and a second stiffness along the handle lateral axis, the first stiffness more than the second stiffness.

57. (canceled)

58. The end effector connector of claim 1, wherein the housing defines an aperture configured for access to the end effector.

59.-136. (canceled)
